(19) United States
(12) Patent Application Publication
(10) Pub. No.: US 2013/0164674 A1
(43) Pub. Date: Jun. 27, 2013

(54) NOVEL ACRYL MONOMER, POLYMER AND RESIST COMPOSITION COMPRISING SAME

(71) Applicant: KOREA KUMHO PETROCHEMICAL CO., LTD., Seoul (KR)

(72) Inventors: Joon Hee HAN, Namyangju-si (KR); Jin Ho KIM, Asan-si (KR); Hyun Soon LIM, Asan-si (KR); Dae Hyeon SHIN, Seoul (KR)

(73) Assignee: KOREA KUMHO PETROCHEMICAL CO., LTD., Seoul (KR)

(21) Appl. No.: 13/724,496

(22) Filed: Dec. 21, 2012

(30) Foreign Application Priority Data
Dec. 23, 2011 (KR) 10-2011-0141173

(51) Int. Cl. G03F 7/039; C08F 224/00; C07C 69/753

(52) U.S. Cl. CPC G03F 7/0397; C07C 69/753; C08F 224/00
USPC 430/270.1; 560/117; 526/268; 430/325; 430/296

(57) ABSTRACT

Disclosed are an acrylic monomer having a structure represented by formula (1), a polymer containing a repeating unit derived from the acrylic monomer, and a resist composition prepared by using the polymer, which exhibits excellent adhesiveness, storage stability, and enhanced line width roughness, exhibits excellent resolution in both C/H patterns and L/S patterns, has an excellent process window so that an excellent pattern profile can be obtained regardless of the type of the substrate, and exhibits improved contrast.

### BACKGROUND OF THE INVENTION

1. Field of the Invention

The present invention relates to a novel acrylic monomer, a polymer, and a resist composition containing the polymer, and more particularly, to a novel acrylic monomer which is useful as a monomer for forming a base polymer of a resist composition which is less dependent on the substrate at the time of resist patterning and is capable of enhancing transparency, contrast, sensitivity, resolution and developability of the resist, a polymer containing a repeating unit derived from the acrylic monomer, and a resist composition containing the polymer.

2. Description of Related Art

Recently, along with high integration of semiconductor devices, there is a demand for the development of a technology for forming ultrafine patterns having a line width of 0.10 micrometers or less in the production of ultra-large scale integrated circuits (LSI) and the like.

Accordingly, the wavelength of the light used in the exposure process has also been further shortened as compared to the region of g-line or i-line that has been conventionally used, and more attention is being paid to studies on lithography using far-infrared radiation, KrF excimer laser light, ArF excimer laser light, extreme ultraviolet laser light (EUV), X-radiation, electron beams and the like. A light source that is attracting the most attention in the field of lithography for forming the next-generation patterns with a line width of 0.10 micrometers or less, is an ArF excimer laser.

A photoresist composition that is generally used in such a fine pattern forming process is composed of a component having an acid-labile functional group (hereinafter, referred to as “polymer”), a component capable of generating an acid when irradiated with a radiation (hereinafter, referred to as “acid generator”), and a solvent, and depending on the cases, a basic additive and the like may also be used.

In the case of the polymer that is used as a primary raw material of photoresists, the polymer should contain functional groups having an appropriate affinity to the developer solution, adhesiveness to the substrate, etching resistance, and excellent resolution power.

Specific examples of such functional groups include a hydroxyl group, a lactone group and a carboxyl group for increasing the affinity to the developer solution, and adhesiveness to the substrate; and cyclic alkyl groups that do not have oxygen atoms in the main chain, such as a norbornene group and an adamantyl group, for enhancing etching resistance. However, in order to increase the resolution, the mobility of the acid generated by the acid generator is more weighted than special functional groups, in view of the structure of the polymer.

Studies have been extensively conducted to date so as to satisfy these properties. Specifically, Korean Patent Applications Laid-Open No. 2001-0104629, No. 2006-0122771, No. 2006-0122773 and No. 2008-0011101 disclose a technology of using a copolymer produced by allowing an acrylic monomer to react with bromoacetyl bromide, and extending the chain length. However, in the current situation, there is an increasing demand for the development of a novel monomer in order to improve the resolution and line edge roughness.

### SUMMARY OF THE INVENTION

An object of the present invention is to provide a novel acrylic monomer which is useful as a monomer for forming a base polymer of a resist composition which is less dependent on the substrate at the time of resist patterning, and is capable of enhancing the transparency, contrast, sensitivity, resolution and developability of the resist.

Another object of the present invention is to provide a polymer containing a repeating unit derived from the acrylic monomer described above,

Still another object of the present invention is to provide a resist composition containing the polymer described above and a method for forming a resist pattern using the resist composition.

According to an aspect of the present invention to achieve the objects described above, there is provided an acrylic monomer represented by the following formula (1):

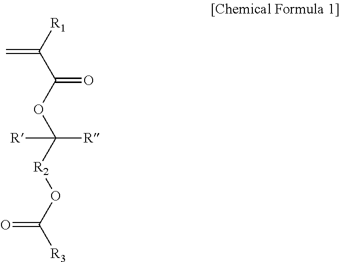

wherein R₁ represents a hydrogen atom or a methyl group;

R₂ represents an alkanediyl group having 1 to 10 carbon atoms;

R₃ represents a cycloalkyl group having 3 to 30 carbon atoms; and

R′ and R″ each independently represent any one selected from the group consisting of a hydrogen atom, an alkyl group having 1 to 10 carbon atoms, a cycloalkyl group having 3 to 30 carbon atoms, and a (C₁-C₁₀ alkyl)cycloalkyl group, provided that R′ and R″ are not hydrogen atoms at the same time.

R₂ may be any one selected from the group consisting of methylene, ethylidene, propylidene, trimethylene, tetramethylene, pentamethylene, hexamethylene, and heptamethylene.

R₃ may be any one selected from the group consisting of a monocyclic cycloalkyl group having 3 to 14 carbon atoms, a bicyclic cycloalkyl group having 8 to 20 carbon atoms, a tricyclic cycloalkyl group having 10 to 30 carbon atoms, and a tetracyclic cycloalkyl group having 10 to 30 carbon atoms.

The acrylic monomer may have a structure of the following formula (1a):

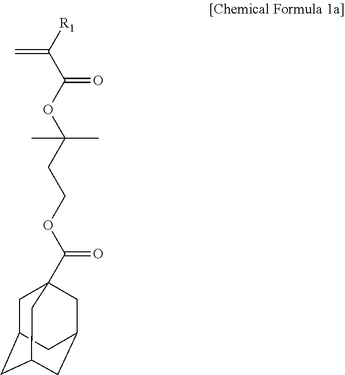

wherein R₁ represents a hydrogen atom or a methyl group.

According to another aspect of the present invention, there is provided a polymer containing a repeating unit represented by the following formula (2):

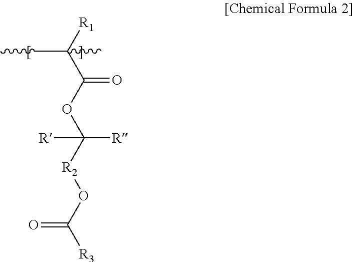

wherein R₁ represents a hydrogen atom or a methyl group;

R₂ represents an alkanediyl group having 1 to 10 carbon atoms;

R₃ represents a cycloalkyl group having 3 to 30 carbon atoms; and

R′ and R″ each independently represent any one selected from the group consisting of a hydrogen atom, an alkyl group having 1 to 10 carbon atoms, a cycloalkyl group having 3 to 30 carbon atom, and a (C₁-C₁₀ alkyl)cycloalkyl group, provided that R′ and R″ are not hydrogen atoms at the same time.

R₂ may be any one selected from the group consisting of methylene, ethylidene, propylidene, trimethylene, tetramethylene, pentamethylene, hexamethylene, and heptamethylene.

R₃ may be any one selected from the group consisting of a monocyclic cycloalkyl group having 3 to 14 carbon atoms, a bicyclic cycloalkyl group having 8 to 20 carbon atoms, a tricyclic cycloalkyl group having 10 to 30 carbon atoms, and a tetracyclic cycloalkyl group having 10 to 30 carbon atoms.

The polymer may contain a repeating unit represented by the following formula (2a):

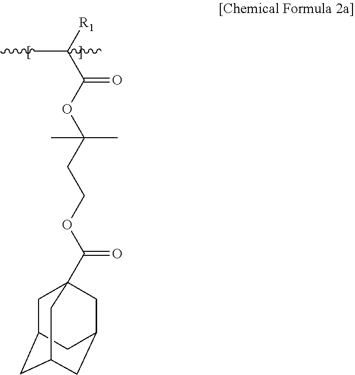

wherein R₁ represents a hydrogen atom or a methyl group.

The polymer may contain one or more repeating units selected from the group consisting of repeating units represented by the following formulae (3) and (4):

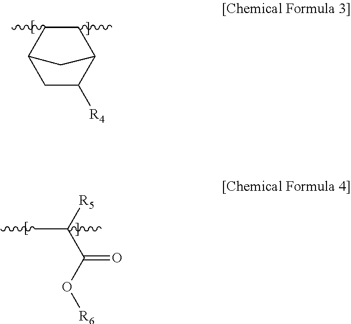

wherein in the above formulae, R₄ and R₆ each independently represent any one selected from the group consisting of a hydrogen atom, an alkyl group having 1 to 10 carbon atoms, a heteroalkyl group having 1 to 10 carbon atoms, a cycloalkyl group having 3 to 30 carbon atoms, an aryl group having 6 to 30 carbon atoms, a heterocyclic group having 2 to 30 carbon atoms, and combinations thereof; and

R₅ represents any one selected from the group consisting of a hydrogen atom, an alkyl group having 1 to 10 carbon atoms, a perfluoroalkyl group having 1 to 10 carbon atoms, and an alkoxy group having 1 to 10 carbon atoms.

The polymer may be represented by the following formula (5):

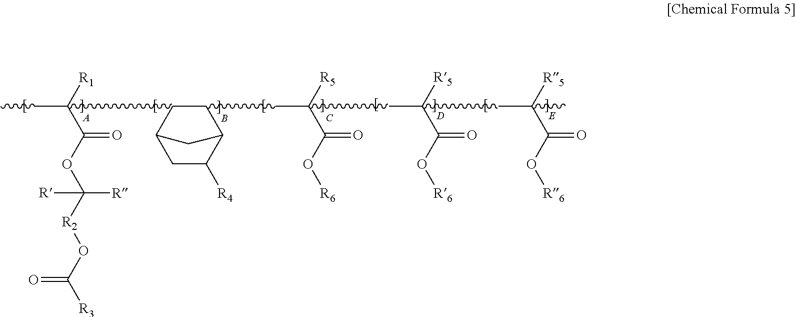

wherein R₁ represents a hydrogen atom or a methyl group;

R₂ represents an alkanediyl group having 1 to 10 carbon atoms;

R₃ represents a cycloalkyl group having 3 to 30 carbon atoms;

R₄ represents any one selected from the group consisting of a hydrogen atom, an alkyl group having 1 to 10 carbon atoms, a heteroalkyl group having 1 to 10 carbon atoms, a cycloalkyl group having 3 to 30 carbon atoms, an aryl group having 6 to 30 carbon atoms, a heterocyclic group having 2 to 30 carbon atoms, and combinations thereof;

R₅, R₅′ and R₅″ each independently represent any one selected from the group consisting of a hydrogen atom, an alkyl group having 1 to 10 carbon atoms, a perfluoroalkyl group having 1 to 10 carbon atoms, and an alkoxy group having 1 to 10 carbon atoms;

R₆, R₆′ and R₆″ each independently represent a hydrogen atom, an alkyl group having 1 to 10 carbon atoms, a heteroalkyl group having 1 to 10 carbon atoms, a cycloalkyl group having 3 to 30 carbon atoms, an aryl group having 6 to 30 carbon atoms, a heterocyclic group having 2 to 30 carbon atoms, and combinations thereof;

R′ and R″ each independently represent any one selected from the group consisting of a hydrogen atom, an alkyl group having 1 to 10 carbon atoms, a cycloalkyl group having 3 to 30 carbon atom, and a (C₁-C₁₀ alkyl)cycloalkyl group, provided that R′ and R″ are not hydrogen atoms at the same time; and

A, B, C, D and E respectively represent the numbers indicating the proportions of the corresponding repeating units in the main chain, and A, B, C, D and E are related such that A+B+C+D+E=1, 0≦A/(A+B+C+D+E)<0.4, 0<B/(A+B+C+D+E)<0.4, 0<C/(A+B+C+D+E)<0.6, 0≦D/(A+B+C+D+E)<0.6, and 0≦E/(A+B+C+D+E)<0.6.

The polymer may contain the repeating unit represented by the formula (2) at a content of 10 mol % to 40 mol %.

The polymer may be selected from the group consisting of polymers represented by the following formulae (6) to (17):

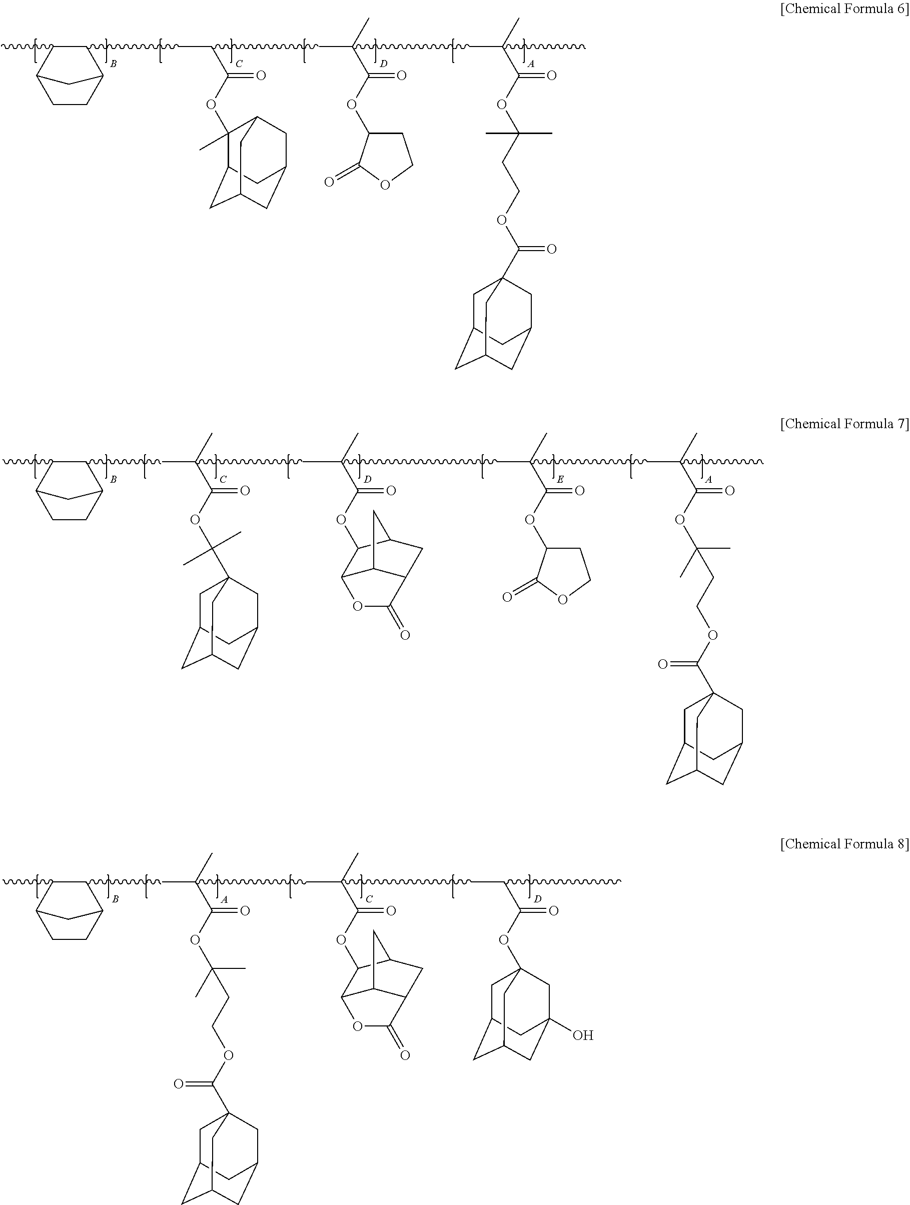

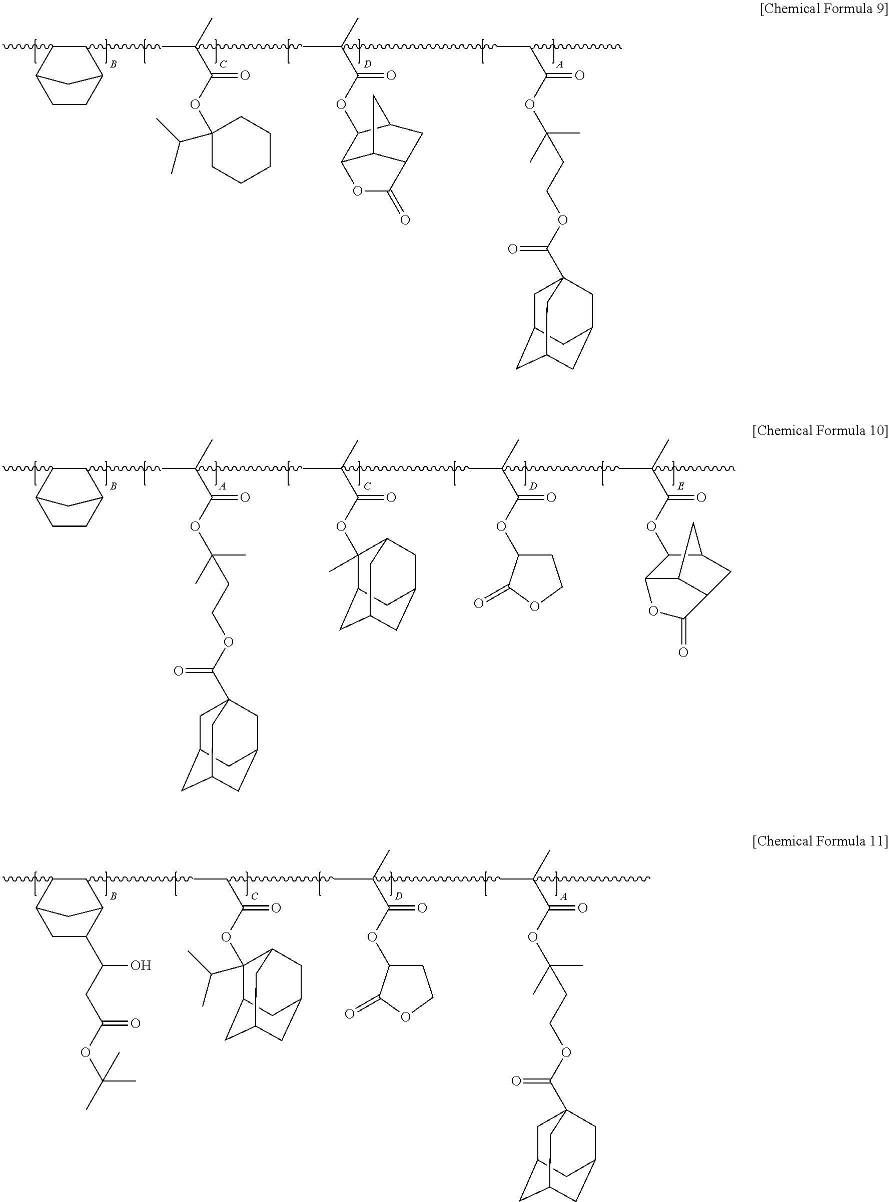

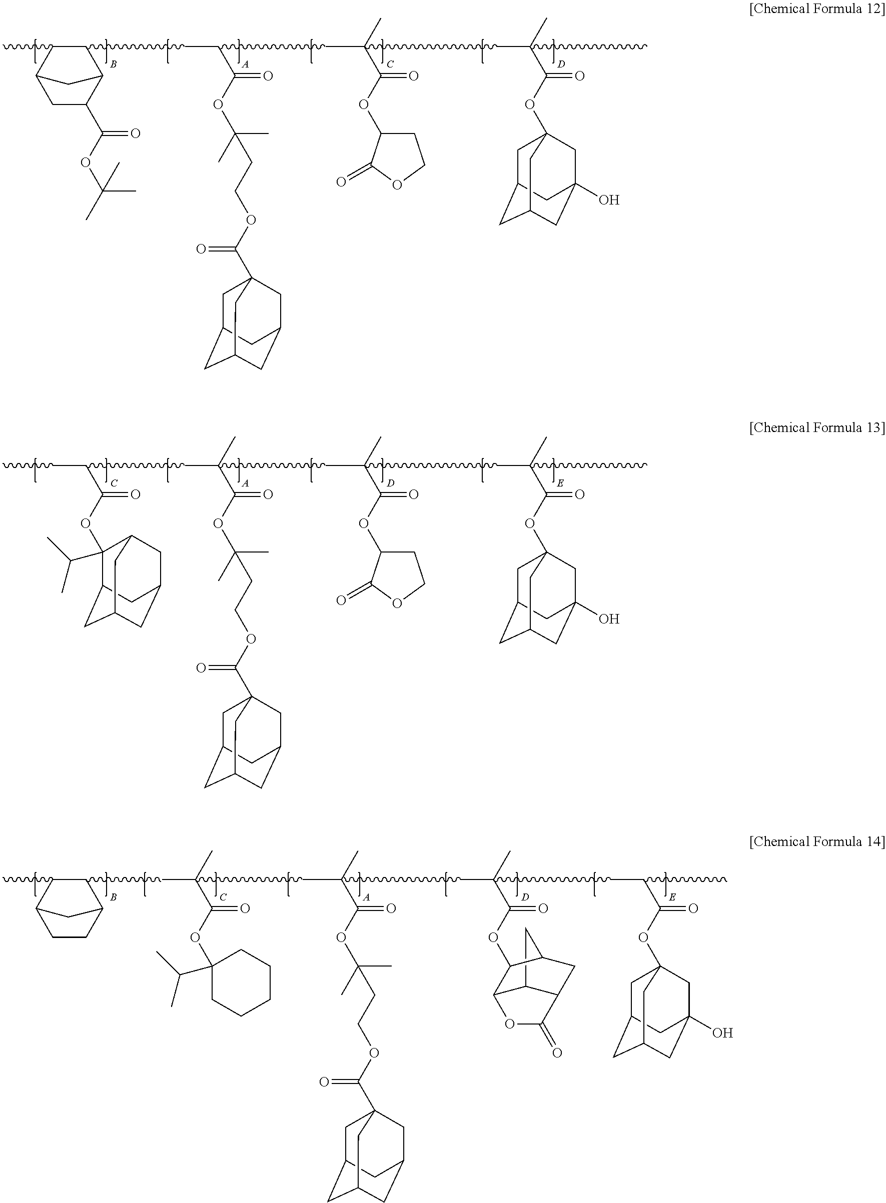

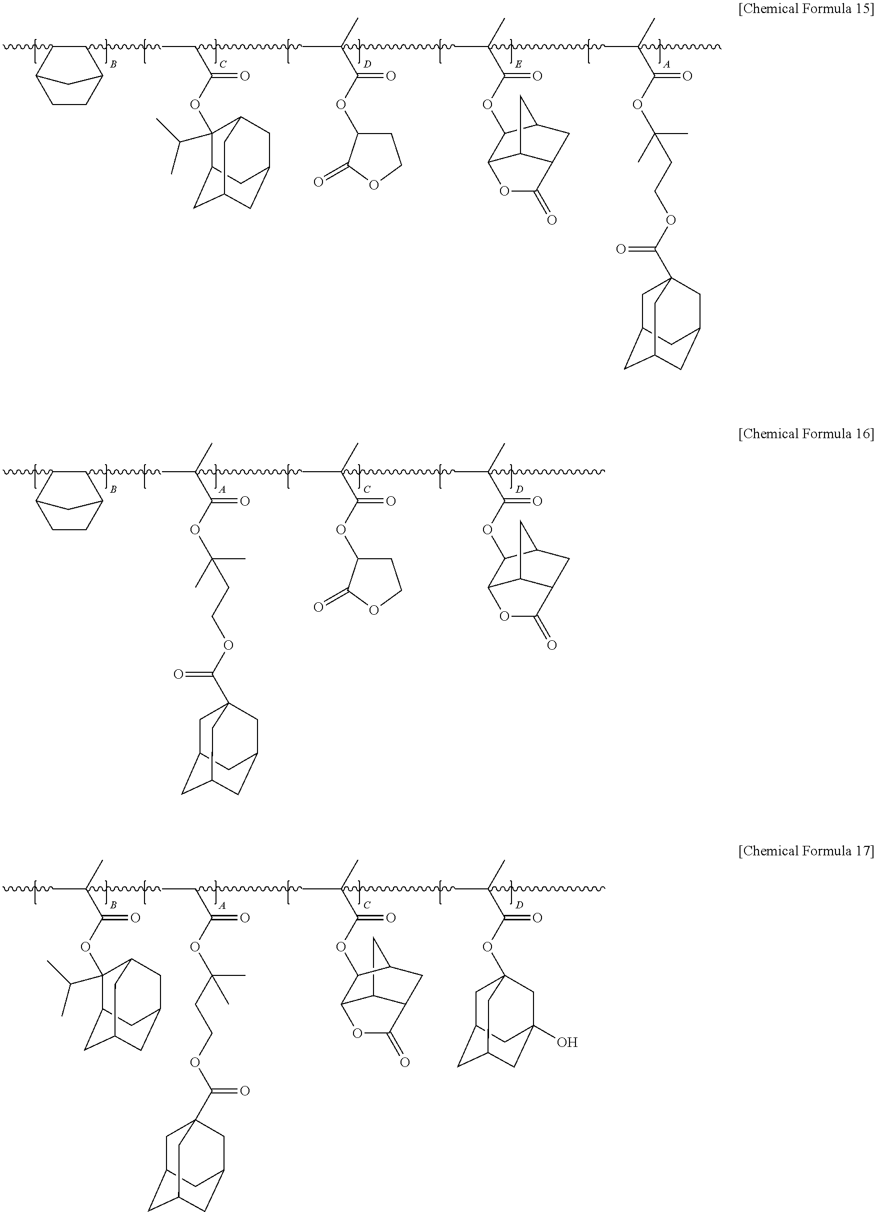

wherein in the above formulae, A, B, C, D and E respectively represent the numbers indicating the proportions of the corresponding repeating units in the main chain, and A, B, C, D and E are related such that A+B+C+D+E=1, 0≦A/(A+B+C+D+E)<0.4, 0<B/(A+B+C+D+E)<0.4, 0<C/(A+B+C+D+E)<0.6, 0≦D/(A+B+C+D+E)<0.6, and 0≦E/(A+B+C+D+E)<0.6.

The polymer may have a weight average molecular weight of 2,000 g/mol to 1,000,000 g/mol as determined by gel permeation chromatography and calculated relative to polystyrene standards, and may have a molecular weight distribution of 1.0 to 5.0.

According to another aspect of the present invention, there is provided a resist composition containing the polymer described above, an acid generator, and a solvent.

The polymer may be included in an amount of 3 wt % to 20 wt % relative to the total weight of the resist composition.

The acid generator may be one or more compounds selected from the group consisting of compounds represented by the following formulae (18) and (19)

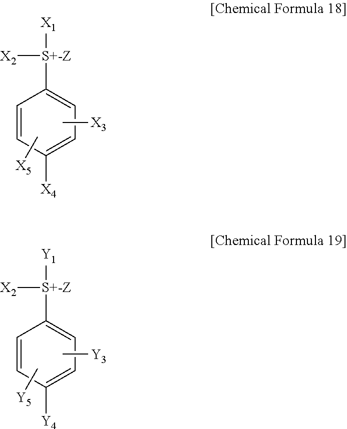

wherein in the formulae, X₁, X₂, Y₁ and Y₂ each independently represent any one selected from the group consisting of a hydrogen atom, an alkyl group having 1 to 10 carbon atoms, an alkenyl group, a haloalkyl group having 1 to 10 carbon atoms, an aralkyl group having 6 to 30 carbon atoms, an aryl group having 6 to 30 carbon atoms, and combinations thereof, while X₁ and X₂, or Y₁ and Y₂ may be joined together to form a saturated or unsaturated hydrocarbon ring having 3 to 30 carbon atoms;

X₃, X₄, X₅, Y₃, Y₄ and Y₅ each independently represent any one selected from the group consisting of a hydrogen atom, an alkyl group having 1 to 30 carbon atoms, a halogen group, an alkoxy group having 1 to 30 carbon atoms, an aryl group having 6 to 30 carbon atoms, a thiophenoxy group, a thioalkoxy group having 1 to 30 carbon atoms, an alkoxycarbonylalkoxy group having 1 to 20 carbon atoms, and combinations thereof;

Z of the anion moiety represents OSO₂CF₃, OSO₂C₄F₉, OSO₂C₈F₁₇, N(CF₃)₂, N(C₂F₅)₂, N(C₄F₉)₂, C(CF₃)₃, C(C₂F₅)₃, C(C₄F₉)₃, or the following formula (20):

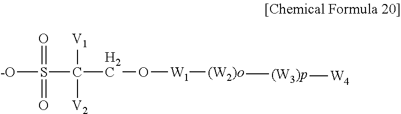

wherein V₁ and V₂ each independently represent a halogen atom;

W₁ represents —(C═O)— or —(SO₂)—;

W₂ represents an alkanediyl group having 1 to 10 carbon atoms;

W₃ represents any one selected from the group consisting of a cycloalkyl group having 3 to 30 carbon atoms, an aryl group having 6 to 30 carbon atoms, an aralkyl group having 7 to 30 carbon atoms, an aryloxy group having 6 to 30 carbon atoms, an arylthio group having 6 to 30 carbon atoms, a heterocyclic group having 5 to 30 carbon atoms, and combinations thereof;

W₄ represents any one selected from the group consisting of a hydrogen atom, a halogen group, an alkyl group having 1 to 10 carbon atoms, an alkoxy group having 1 to 10 carbon atoms, a haloalkyl group having 1 to 10 carbon atoms, an alkylthio group having 1 to 10 carbon atoms, an aryl group having 6 to 30 carbon atoms, and combinations thereof;

o represents an integer of 0 or 1; and

p represents an integer from 0 to 2.

The acid generator may be included in an amount of 0.3 to 10 parts by weight relative to 100 parts by weight of the solids content in the resist composition.

The resist composition may further include additives selected from the group consisting of an alkali dissolution inhibitor, an acid diffusion inhibitor, a surfactant, and mixtures thereof.

According to another aspect of the present invention, there is provided a method for forming a resist pattern, which includes a step of applying the resist composition described above on a substrate and thereby forming a resist film; a step of heat treating the resist film and then exposing the resist film into a predetermined pattern; and a step of developing the exposed resist pattern.

The exposure process may be carried out by using a light source selected from the group consisting of a KrF excimer laser, an ArF excimer laser, an extreme ultraviolet laser, X-radiation, and an electron beam.

The details of other embodiments of the present invention will be disclosed in the detailed description of the invention given below.

The acrylic monomer according to the present invention is useful as a monomer for forming a base polymer of a resist composition, particularly a positive chemically amplified photoresist composition, which is less dependent on the substrate at the time of resist patterning, and is capable of enhancing the transparency, contrast, sensitivity, resolution and developability of the resist.

The polymer according to the present invention containing a repeating unit derived from the monomer described above is useful for the formation of a fine pattern using various radiations such as far-ultraviolet radiation of a KrF excimer laser, an ArF excimer laser or a F₂ excimer laser; X-radiation such as synchrotron radiation; and charged particle beams such as EUV.

Furthermore, the resist composition according to the present invention containing the polymer described above exhibits excellent adhesiveness, storage stability and enhanced line width roughness, and exhibits excellent resolution in both C/H patterns and L/S patterns.

Also, since the resist composition has excellent process window, an excellent pattern profile can be obtained regardless of the type of the substrate, and improved contrast is exhibited.

### BRIEF DESCRIPTION OF THE DRAWINGS

FIG. 1 is a proton nuclear magnetic resonance (¹H-NMR (CDCl₃, 400 MHz)) spectrum of the intermediate (iii) produced in Step 1 of Synthesis Example 1 for the acrylic monomer.

FIG. 2 is a ¹H-NMR (CDCl₃, 400 MHz) spectrum of the compound of formula (1a) produced in Step 2 of Synthesis Example 1 for the acrylic monomer.

FIG. 3 is a ¹H-NMR (CDCl₃, 400 MHz) spectrum of the polymer (16) produced in Intermediate Synthesis Example 1.

FIG. 4 is a ¹H-NMR (CDCl₃, 400 MHz) spectrum of the polymer (15) produced in Polymer Synthesis Example 2.

### DETAILED DESCRIPTION OF THE INVENTION

Hereinafter, embodiments of the present invention will be described in detail. However, these embodiments are only for illustrative purposes, and the present invention is not intended to be limited thereto. The present invention is to be defined only by the scope of the claims that will be described below.

Unless particularly stated otherwise in the present specification, the term “halogen atom” means any one selected from the group consisting of fluorine, chlorine, bromine and iodine.

Unless particularly stated otherwise herein, the prefix “hetero-” means that one to three carbon atoms are substituted by heteroatoms selected from the group consisting of nitrogen (N), oxygen (O), sulfur (S) and phosphorus (P). For example, a heteroalkyl group means that one to three carbon atoms among the carbon atoms of an alkyl group are substituted by heteroatoms.

Unless particularly stated otherwise herein, the term “alkyl group” means a linear or branched alkyl group having 1 to 30 carbon atoms, and the alkyl group includes a primary alkyl group, a secondary alkyl group and a tertiary alkyl group.

Unless particularly stated otherwise herein, the term “alkanediyl” means a divalent atomic group obtained by removing two hydrogen atoms from an alkane, and may be represented by the formula: —CₙH₂ₙ—.

Unless particularly stated otherwise herein, the term “alkenyl” means a hydrocarbon having one or more unsaturated regions, that is, normal, secondary, tertiary or cyclic carbon atoms having carbon-carbon, sp₂ double bonds. For example, an alkenyl group may have 2 to 20 carbon atoms (that is, C₂-C₂₀ alkenyl), 2 to 12 carbon atoms (that is, C₂-C₁₂ alkenyl), or 2 to 6 carbon atoms (C₂-C₆ alkenyl). Suitable examples of the alkenyl group include, but are not limited to, ethylene or vinyl (—CH═CH₂), allyl (—CH₂CH═CH₂), cyclopentenyl (—C₅H₇), and 5-hexenyl (—CH₂CH₂CH₂CH₂CH═CH₂).

Unless particularly stated otherwise herein, the term “haloalkyl” means an alkyl group in which one or more hydrogen atoms of an alkyl group such as defined above are substituted by halogen atoms. The alkyl moiety of the haloalkyl group may have 1 to 20 carbon atoms (that is, C₁-C₂₀ haloalkyl), 1 to 12 carbon atoms (that is, C₁-C₁₂ haloalkyl), or 1 to 6 carbon atoms (that is, C₁-C₆ alkyl). Suitable examples of the haloalkyl group include, but not limited to, —CF₃, —CHF₂, —CFH₂, —CH₂CF₃, and perfluoroalkyl.

Unless particularly stated otherwise herein, the term “perfluoroalkyl group” means an alkyl group having 1 to 10 carbon atoms, in which some or all of the hydrogen atoms have been substituted by fluorine atoms.

Unless particularly stated otherwise herein, the term “alkoxy” means an —ORa group, wherein Ra means an alkyl such as defined above. Examples of an alkoxy group useful for the present invention include, but are not limited to, methoxy, difluoromethoxy, trifluoromethoxy, ethoxy, n-propoxy, isopropoxy, n-butoxy, and t-butoxy.

Unless particularly stated otherwise here, the term “perfluoroalkoxy group” means an alkoxy group having 1 to 10 carbon atoms, in which some or all of the hydrogen atoms are substituted by fluorine atoms.

Unless particularly stated otherwise herein, the term “cycloalkyl group” means a cycloalkyl group having 3 to 30 carbon atoms, and includes monocyclic, bicyclic, tricyclic and tetracyclic alkyl groups.

Furthermore, the cycloalkyl group includes an adamantyl group, a norbornyl group, and a polycyclic cycloalkyl group containing a norbornyl group.

Unless particularly stated otherwise herein, the aryl group means a compound containing a benzene ring or a derivative thereof, and examples include a compound in which an alkyl side chain is linked to a benzene ring, such as toluene or xylene; a compound in which two or more benzene rings are linked via a single bond, such as biphenyl; a compound in which two or more benzene rings are linked via a cycloalkyl group or a heterocycloalkyl group, such as fluorene, xanthene or anthraquinone; and a compound in which two or more benzene rings are fused together, such as naphthalene or anthracene. Unless particularly stated otherwise here, the aryl group means an aryl group having 6 to 30 carbon atoms.

Unless particularly stated otherwise here, the term “aralkyl” means an alkyl substituted with an aryl group having 6 to 30 carbon atoms such as defined above, and the alkyl and the aryl have the same meanings as defined above. Examples of the aralkyl include benzyl, phenethyl, and phenylpropyl.

Unless particularly stated otherwise herein, the term “aryloxy” means an —ORb group, wherein Rb represents an aryl such as defined above. Examples of an aryloxy group useful for the present invention include, but are not limited to, a phenoxy group.

Unless particularly stated otherwise herein, the term “arylthio” means an —SRc group, wherein Re represents an aryl such as defined above. Examples of an arylthio group useful for the present invention include, but are not limited to, a phenylthio group.

Unless particularly stated otherwise herein, the term “alkylthio” means an —SRd group, wherein Rd represents an alkyl such as defined above. Examples of an alkylthio group useful for the present invention include, but are not limited to, methylthio, ethylthio, n-propylthio, isopropylthio, n-butylthio and t-butylthio.

Unless particularly stated otherwise herein, the term “heterocyclic group” means a cyclic radical having 4 to 20 ring-constituting atoms, in which one or more (for example, 1, 2, 3 or 4) carbon atoms are substituted by heteroatoms (for example, N, O, P or S). The term “heterocyclic group” includes a saturated ring, a partially unsaturated ring, and an aromatic ring (that is, a heteroaromatic ring), and also includes a cyclic aromatic radical in which the heteroatoms in the ring are oxidized or quaternized to form, for example, an N-oxide or a quaternary salt. Examples of a substituted heterocyclic group include heterocyclic rings substituted with any arbitrary substituent disclosed herein, including a carbonyl group.

Examples of the heterocyclic group include, but are not limited to, pyridyl, dihydropyridyl, tetrahydropyridyl (piperidyl), thiazolyl, tetrahydrothiophenyl, tetrahydrothiophenyl sulfate, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyradinyl, pyridazinyl, indolidinyl, isoindolyl, 3H-indolyl, 1H-indazolyl, purinyl, 4H-quinolidinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, bistetrahydrofuranyl (each of these may be substituted or unsubstituted), and N-oxides (for example, pyridyl N-oxide and quinolinyl N-oxide) and quaternary salts thereof.

Unless particularly stated otherwise, all the compounds and substituents mentioned in the present specification may be substituted or unsubstituted. Here, the term “substituted” means that a hydrogen atom is substituted by any one selected from the group consisting of a halogen atom, a hydroxyl group, a carboxyl group, a cyano group, a nitro group, an amino group, a thio group, a methylthio group, an alkoxy group, a nitrile group, an aldehyde group, an epoxy group, an ether group, an ester group, a carbonyl group, an acetal group, a ketone group, an alkyl group, a perfluoroalkyl group, a cycloalkyl group, a heterocycloalkyl group, an allyl group, a benzyl group, an aryl group, a heteroaryl group, derivatives thereof, and combinations thereof.

Furthermore, unless particularly stated otherwise, the term “combinations thereof” according to the present specification means that two or more substituents are linked by a single bond or a linking group, or two or more substituents are linked by condensation.

Recently, along with high integration of semiconductor devices, there is a demand for a technology for forming ultrafine patterns having a line width of 0.10 micrometers or less in the production of ultra-LSI and the like, and as ArF immersion lithography is in progress, there is a demand for a technology for forming patterns having a line width of 0.05 micrometers or less. Therefore, in order to realize line width that are becoming increasingly finer, attention is being paid to the study on lithography of using a short wavelength as the exposure wavelength, and using EUV using a wavelength of 13.5 nm, as a next generation light of ArF light that uses a wavelength of 193 nm. The principle related to this may be understood from Rayleigh's equation: R=kλ/NA². However, high resolution is not a property obtainable simply by reducing the wavelength of light, and while patterns become finer and finer, the resolution may be determined by the degree of roughness of the pattern developed with a developer solution. Accordingly, as one of the measures to lower the line edge roughness, research is conducted on a method of improving the line edge roughness by increasing the carbon number of the pendant group attached to the main chain of the polymer used in the resist, and thereby extending the chain, as compared with existing monomers.

In the present invention, it was found that, in order to increase the carbon number in the group that is linked to the main chain of the polymer for resist, when an alkanediol, for example, 3-methyl-1,3-butanediol is used to react with an acid-labile group-containing compound such as adamantanol or adamantanecarboxylic acid, and then the product is allowed to react with a (meth)acryl halide to synthesize a novel acrylic monomer, and a polymer produced by using this acrylic monomer is used as a base resin for resist, the line edge roughness in the immersion exposure system using ArF is improved, and at the time of irradiation with ArF laser light, an adamantanyloxydimethylpropyl group is detached and separated from the group linked to the main chain of the polymer under the action of an acid. It was also found that thereby, fine patterns can be realized, and the developability by a developer solution and the adhesive power to the substrate are improved, and thus the present invention was completed.

That is, the acrylic monomer according to an embodiment of the present invention has a structure of the following formula (1):

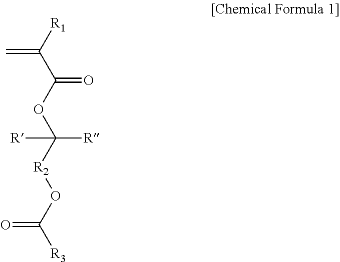

wherein R₁ represents a hydrogen atom or a methyl group;

R₂ represents an alkanediyl group having 1 to 10 carbon atoms;

R₃ represents a cycloalkyl group having 3 to 30 carbon atoms;

R′ and R″ each independently represent any one selected from the group consisting of a hydrogen atom, an alkyl group having 1 to 10 carbon atoms, a cycloalkyl group having 3 to 30 carbon atoms, and a (C₁-C₁₀ alkyl)cycloalkyl group, provided that R′ and R″ are not hydrogen atoms at the same time.

Specifically, R₂ in the formula (1) may be any one selected from the group consisting of methylene, ethylidene, propylidene, trimethylene, tetramethylene, pentamethylene, hexamethylene, and heptamethylene.

Furthermore, R₃ in the formula (1) may be any one selected from the group consisting of a monocyclic cycloalkyl group having 3 to 14 carbon atoms, a bicyclic cycloalkyl group having 8 to 20 carbon atoms, a tricyclic cycloalkyl group having 10 to 30 carbon atoms, and a tetracyclic cycloalkyl group having 10 to 30 carbon atoms, and 1 to 5 hydrogen atoms among the hydrogen atoms of R₃ may be substituted by any one selected from the group consisting of an alkyl group having 1 to 6 carbon atoms, an alkoxy group having 1 to 6 carbon atoms, a perfluoroalkyl group having 1 to 4 carbon atoms, a perfluoroalkoxy group having 1 to 4 carbon atoms, a hydroxyalkyl group having 1 to 6 carbon atoms, a halogen atom, a hydroxyl group, a carboxyl group, a cyano group, a nitro group, an amino group, a thio group, a methylthio group, a methoxy group, OR′″, COR′″ and COOR′″, while R′″ may be any one selected from the group consisting of an alkyl group and an aryl group.

Preferably, R₃ may be any one selected from the group consisting of atomic groups represented by the following formulae (1-1) to (1-9):

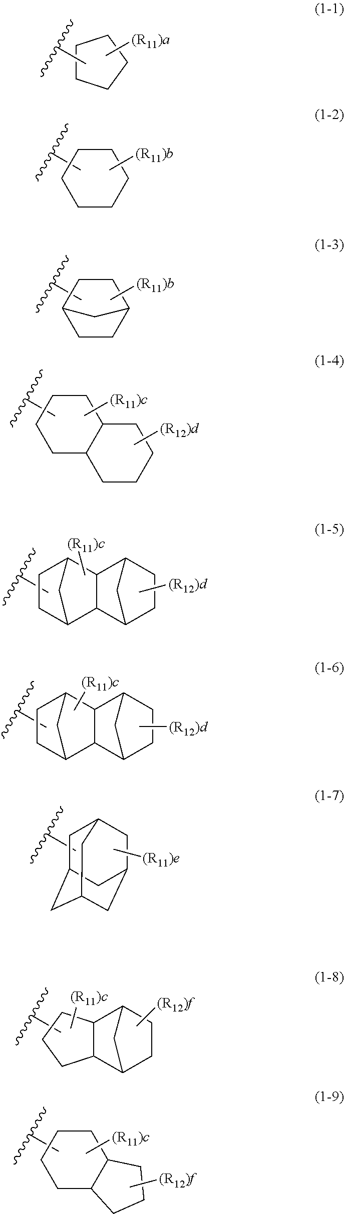

In the above formulae (1-1) to (1-9), R₁₁ and R₁₂ each independently represent hydrogen atom, an alkyl group having 1 to 6 carbon atoms, an alkoxy group having 1 to 6 carbon atoms, a perfluoroalkyl group having 1 to 4 carbon atoms, a perfluoroalkoxy group having 1 to 4 carbon atoms, a hydroxyalkyl group having 1 to 6 carbon atoms, a halogen atom, a hydroxyl group, a carboxyl group, a cyano group, a nitro group, an amino group, a thio group, a methylthio group, a methoxy group, OR′″, COR′″ and COOR′″, while R′″ may be any one selected from the group consisting of any one selected from the group consisting of an alkyl group and an aryl group;

a, c and d are each independently represent an integer from 0 to 9, b represents an integer from 0 to 11, e represents an integer from 0 to 15, f represents an integer from 0 to 7, 0≦c+d≦17, and 0≦c+f≦15.

More preferably, R₃ represents an adamantyl group or a norbornyl group.

In the formula (1), R′ and R″ are preferably each independently selected from the group consisting of a hydrogen atom, an alkyl group having 1 to 5 carbon atoms, a monocyclic cycloalkyl group having 3 to 14 carbon atoms, a bicyclic cycloalkyl group having 8 to 20 carbon atoms, a tricyclic cycloalkyl group having 10 to 30 carbon atoms, a tetracyclic cycloalkyl group having 10 to 30 carbon atoms, and a (C₁-C₅ alkyl)cycloalkyl group, provided that R′ and R″ are not hydrogen atoms at the same time.

Preferably, R′ and R″ are each independently a methyl group or an ethyl group.

Preferred examples of the acrylic monomer according to the present invention include adamantane-1-carboxylic acid 3-methyl-3-(2-methylacryloyloxy)butyl ester (hereinafter, referred to as IAM-1) represented by the following formula (1a), and adamantane-1-carboxylic acid 3-methyl-3-acryloyloxybutyl ester.

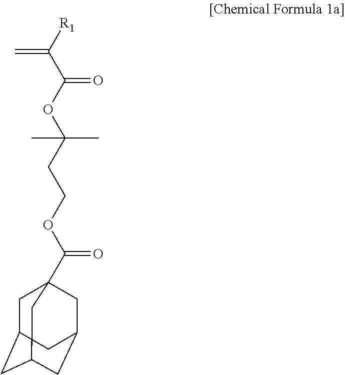

wherein R₁ represents a hydrogen atom or a methyl group.

An acrylic monomer having a structure such as described above can be produced by allowing an alkanediol to react with an acid-labile group-providing compound, and then allowing the compound thus obtained to react with a (meth)acrylic halide.

At this time, an alkanediol having 3 to 20 carbon atoms containing a functional group having a tertiary structure in the molecule may be used as the alkanediol, and specifically, 3-methyl-1,3-butanediol or the like can be used.

As the acid-labile group-providing compound, any one selected from the group consisting of an alcohol, a carboxylic acid and a halide, all of which contain an acid-labile group, and compounds thereof may be used, and the acid-labile group has the same meaning as defined above. Specifically, a compound selected from the group consisting of adamantanol, adamantanecarboxylic acid, adamantanecarbonyl chloride, and mixtures thereof can be used.

Furthermore, as the (meth)acrylic halide, any one selected from the group consisting of methacrylic chloride, acrylic chloride, and mixtures thereof can be used.

The above-mentioned compounds used in the production of the acrylic monomer according to the present invention can be used in their stoichiometric amounts.

The acrylic monomer according to the present invention produced by a method such as described above is less dependent on the substrate at the time of resist patterning, and can improve the transparency, contrast, sensitivity, resolution and developability of the resist. Thus, the acrylic monomer is useful as a monomer for forming a base polymer for a resist composition, particularly a positive chemically amplified photoresist composition.

Thus, according to another embodiment of the present invention, there is provided a polymer containing a repeating unit that is derived from the acrylic monomer.

More particularly, the polymer according to the present invention contains a repeating unit represented by the following formula (2), which is derived from the acrylic monomer described above.

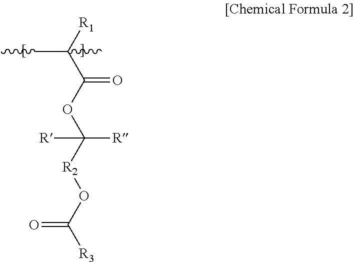

wherein R₁ to R₃, and R′ and R″ respectively have the same meanings as defined above.

Preferably, the polymer may contain a repeating unit represented by the following formula (2a):

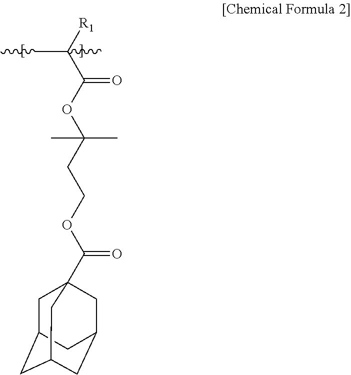

wherein R₁ has the same meaning as defined above.

The polymer according to the present invention may further contain, together with the acrylic monomer of the above formula (1), one or more repeating units represented by the following formulae (3) and (4), which are derived from a (meth)acrylate monomer and a norbornene derivative.

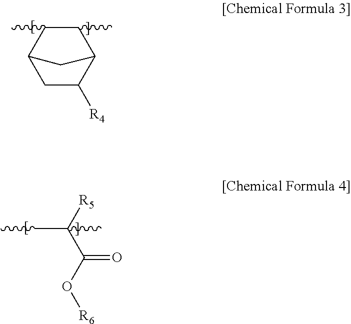

wherein in the formulae shown above,

R₄ and R₆ each independently represent any one selected from the group consisting of a hydrogen atom, an alkyl group having 1 to 10 carbon atoms, a heteroalkyl group having 1 to 10 carbon atoms, a cycloalkyl group having 3 to 30 carbon atoms, an aryl group having 6 to 30 carbon atoms, a heterocyclic group having 2 to 30 carbon atoms, and combinations thereof; and

R₅ represents any one selected from the group consisting of a hydrogen atom, an alkyl group having 1 to 10 carbon atoms, a perfluoroalkyl group having 1 to 10 carbon atoms, and an alkoxy group having 1 to 10 carbon atoms.

Specifically, the polymer may be a multi-component polymer represented by the following formula (5), and the linking sequence of the respective repeating units in the structure is subject to change.

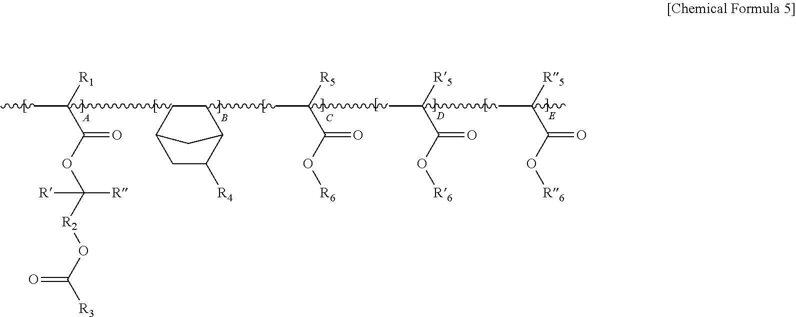

wherein R₁ represents a hydrogen atom or a methyl group;

R₂ represents an alkanediyl group having 1 to 10 carbon atoms;

R₃ represents a cycloalkyl group having 3 to 30 carbon atoms;

R₄ represents any one selected from the group consisting of a hydrogen atom, an alkyl group having 1 to 10 carbon atoms, a heteroalkyl group having 1 to 10 carbon atoms, a cycloalkyl group having 3 to 30 carbon atoms, an aryl group having 6 to 30 carbon atoms, a heterocyclic group having 2 to 30 carbon atoms, and combinations thereof;

R₅, R₅′ and R₅″ each independently represent any one selected from the group consisting of a hydrogen atom, an alkyl group having 1 to 10 carbon atoms, a perfluoroalkyl group having 1 to 10 carbon atoms, and an alkoxy group having 1 to 10 carbon atoms;

R₆, R₆′ and R₆″ each independently represent any one selected from the group consisting of a hydrogen atom, an alkyl group having 1 to 10 carbon atoms, a heteroalkyl group having 1 to 10 carbon atoms, a cycloalkyl group having 3 to 30 carbon atoms, an aryl group having 6 to 30 carbon atoms, a heterocyclic group having 2 to 30 carbon atoms, and combinations thereof;

R′ and R″ each independently represent any one selected from the group consisting of a hydrogen atom, an alkyl group having 1 to 10 carbon atoms, a cycloalkyl group having 3 to 30 carbon atoms, and a (C₁-C₁₀ alkyl)cycloalkyl group, provided that R′ and R″ are not hydrogen atoms at the same time;

A, B, C, D and E respectively represent the numbers indicating the proportions of the corresponding repeating units in the main chain, and A, B, C, D and E are related such that A+B+C+D+E=1, 0≦A/(A+B+C+D+E)<0.4, 0<B/(A+B+C+D+E)<0.4, 0<C/(A+B+C+D+E)<0.6, 0≦D/(A+B+C+D+E)<0.6, and 0≦E/(A+B+C+D+E)<0.6.

In the formula (5), R₄, R₆, R₆′ and R₆″ may be each independently any one selected from the group consisting of atomic groups represented by the following formulae (5-1) to (5-12).

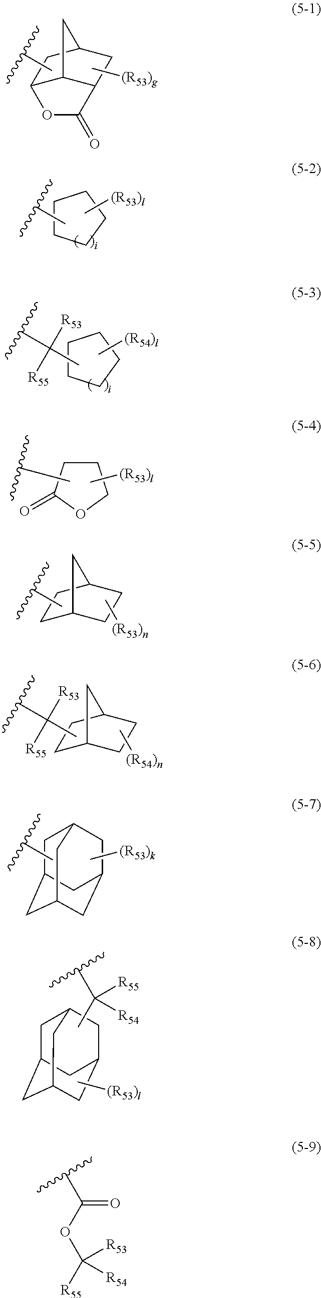

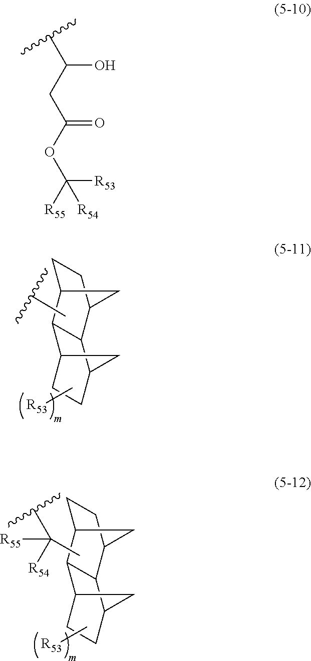

In the formulae (5-1) to (5-12),

R₅₃ to R₅₅ each independently represent any one selected from the group consisting of a hydrogen atom, an alkyl group having 1 to 6 carbon atoms, an alkoxy group having 1 to 6 carbon atoms, a perfluoroalkyl group having 1 to 4 carbon atoms, a perfluoroalkoxy group having 1 to 4 carbon atoms, a hydroxyalkyl group having 1 to 6 carbon atoms, a halogen atom, a hydroxyl group, a carboxyl group, a cyano group, a nitro group, an amino group, a thio group, a methylthio group, and a methoxy group;

g represents an integer from 0 to 9; h represents an integer from 0 to 9; i represents an integer from 0 to 4; j represents an integer from 0 to 5; k represents an integer from 0 to 15; I represents an integer from 0 to 15; m represents an integer from 0 to 17; and n represents an integer from 0 to 11.

More preferably, R₄, R₆, R₆′ and R₆″ in the formula (5) may be selected from the group consisting of atomic groups represented by the formulae (5-3), (5-4), (5-6), (5-8) to (5-10) and (5-12).

Furthermore, in the formula (5), R₅, R₅′ and R₅″ are each independently a hydrogen atom or a methyl group.

In the polymer of the formula (5), repeating unit A undergoes activation of the carbonyl group by the acid generated at the time of light irradiation, and as a result, a hydrogen atom is detached from the tertiary-form functional group that is boned to the oxygen atom of a (meth)acrylic acid group, for example, a tert-butanediyl group. Thereby, the repeating unit A is changed to an isoprene form, and a liberation reaction occurs. At this time, if the molecular weight of the functional group that is liberated is small, that is, if the number of carbon atoms is small, the polymer may be vaporized at the time of post-exposure after the exposure, causing contamination of the lens of the exposure machine, and the vaporized polymer may cause a thickness loss or defects at the time of a heat treatment of the resist film. However, in the present invention, the functional group that is liberated has an appropriate molecular weight and is not vaporized. Furthermore, even if the liberated functional group remains in the resist, the liberated functional group does not affect the pattern profile, and may be developed together by a developer solution at the time of the development of exposed areas. As a result, the line edge roughness is improved, and the realization of fine patterns is made possible.

Preferably, it is preferable for the polymer according to the present invention to include the repeating unit A at a content of 10 mol % to 40 mol %, from the viewpoint that excellent pattern forming properties can be exhibited even with a smaller amount of the photoacid generator. If the content of the repeating unit A is out of the range described above, a change in polarity does not occur to the extent that sufficient solubility in an aqueous alkali solution may be obtained, and there is a risk that realization of patterns may not be achieved.

Furthermore, the repeating unit A can satisfy the sensitivity that is required in resists, when used together with a highly sensitive acid-labile group. Therefore, an effect that line edge roughness at the boundary between an unexposed area and an exposed area that are distinguished by the developer solution can be prevented may be obtained.

Since the repeating unit derived from a norbornene derivative represented by the repeating unit B in the polymer of the formula (5) has a characteristic of deriving the resulting polymer into a copolymer having a deformed helical structure, the low solubility in solvents exhibited by conventional methacrylic copolymers is improved. Furthermore, since a repeating unit that is derived from a norbornene derivative having a structure such as described above plays the role as a molecular weight adjusting agent, a low molecular weight polymer can be produced by adjusting the degree of polymerization of the acrylic monomer, and etching resistance can be enhanced.

Furthermore, in the acrylic derivatives in the repeating units C, D and E of the polymer of the formula (5), acid-labile functional groups can be introduced, and a moiety related to an improvement of adhesive power for reinforcing the adhesive power on the wafer can also be introduced. It is general to use an acrylic monomer mainly containing lactone as such an adhesive power reinforcing agent. More preferably, a functional group containing lactone for reinforcing the adhesive power between the acid-labile functional group and the substrate is used, and also, a bulky hydrocarbon compound which increases resistance to etching can be simultaneously introduced.

The copolymer according to the present invention having such a structure as described above may be a block copolymer, a random copolymer, or a graft copolymer.

Specific examples of the polymer according to the present invention include compounds having the structure of the following formulae (6) to (17), and the sequence of the respective repeating units in the structural formulae is subject to change:

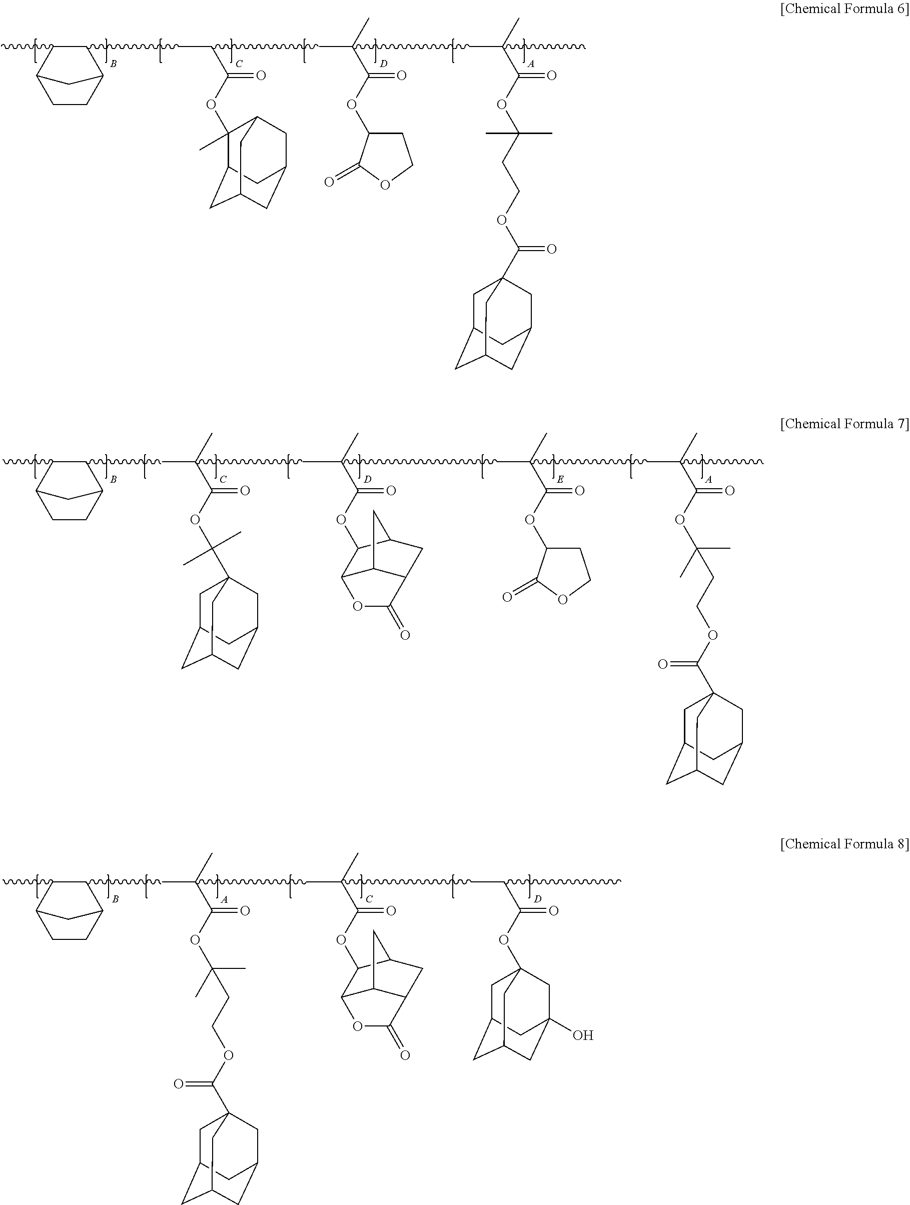

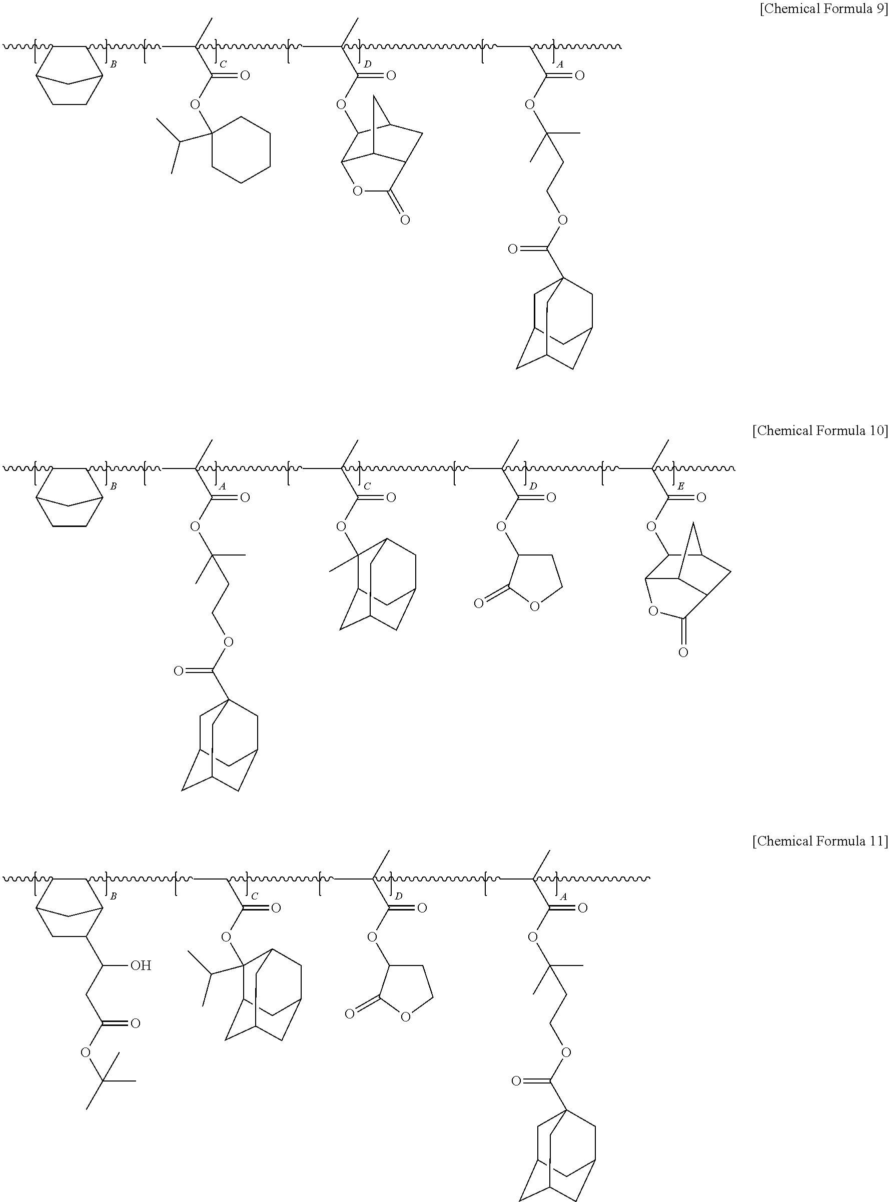

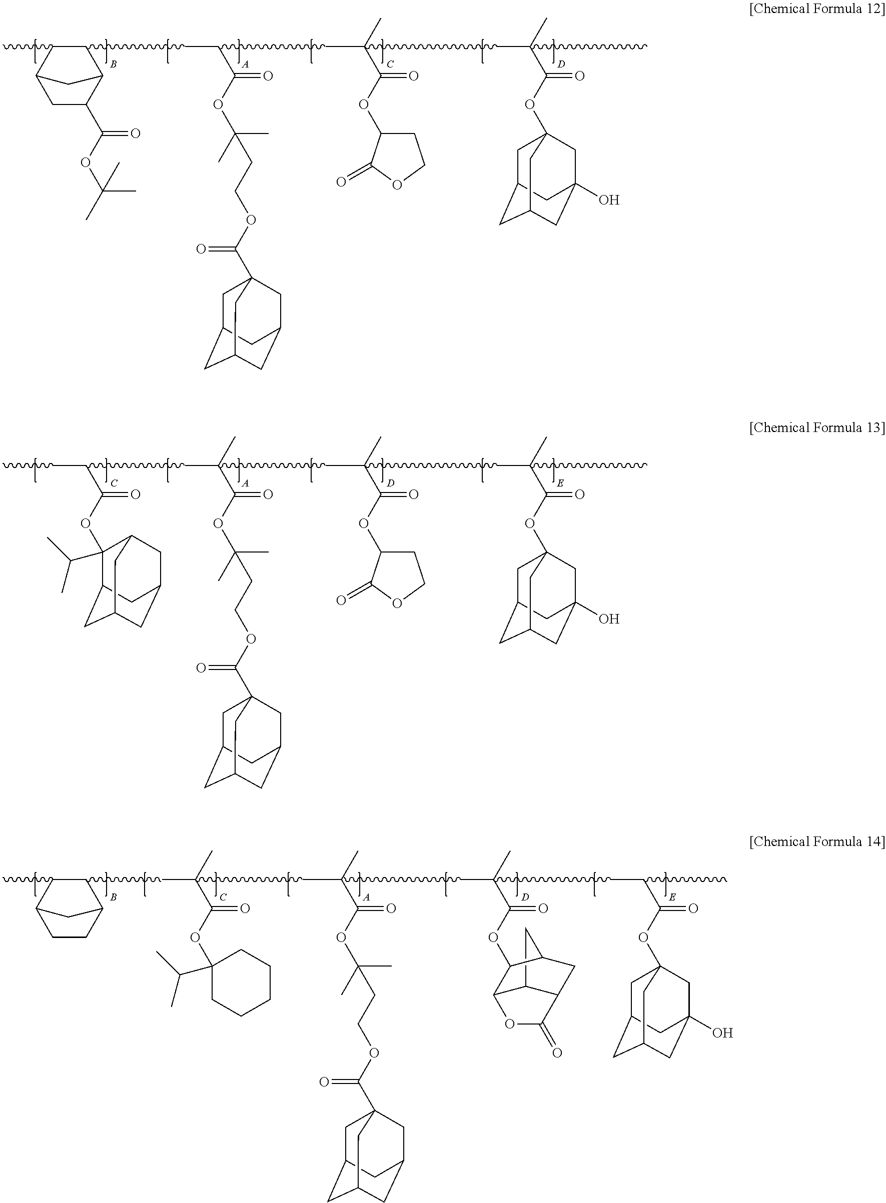

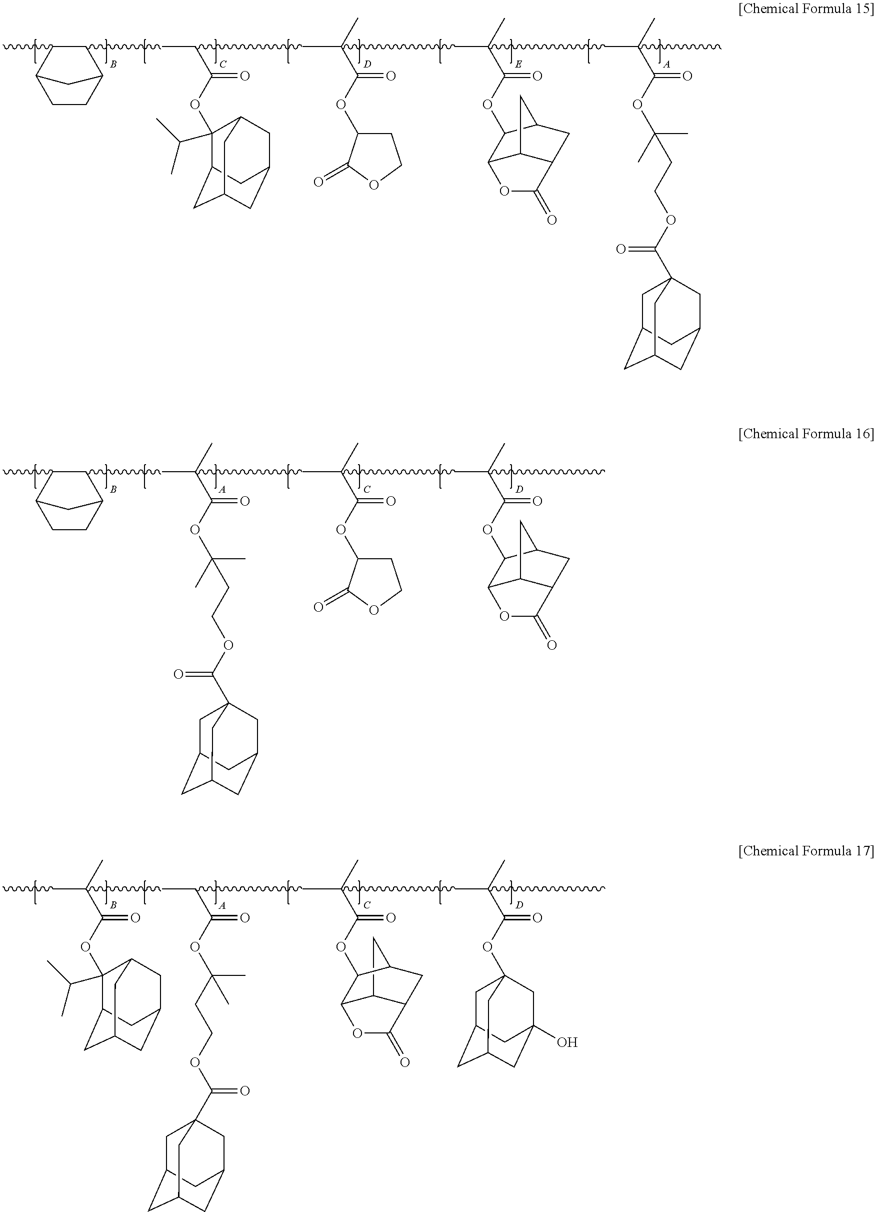

wherein in the above formulae, A, B, C, D and E respectively represent the numbers indicating the proportions of the corresponding repeating units in the main chain, and A, B, C, D and E are related such that A+B+C+D+E=1, 0≦A/(A+B+C+D+E)<0.4, 0<B/(A+B+C+D+E)<0.4, 0<C/(A+B+C+D+E)<0.6, 0≦D/(A+B+C+D+E)<0.6, and 0≦E/(A+B+C+D+E)<0.6.

The polymer according to the present invention is generally insoluble or sparingly soluble per se in an aqueous alkali solution, but depending on the cases, the polymer may be soluble.

Also, the polymer according to the present invention may have increased or decreased solubility depending on the change in the type and content of the monomer in the polymer. In general, as the number of hydrophobic groups increases, the solubility in an aqueous alkali solution decreases.

The polymer according to the present invention is such that the weight average molecular weight (hereinafter, referred to as “Mw”) determined by gel permeation chromatography (GPC) and calculated relative to polystyrene standards is preferably 2,000 g/mol to 1,000,000 g/mol, and in view of the sensitivity, developability, coatability and heat resistance of the resist at the time when the polymer is used as a base resin for resist, the weight average molecular weight is more preferably 3,000 g/mol to 50,000 g/mol. Furthermore, for a reduction of the line edge roughness, the polymer preferably has a molecular weight distribution of 1 to 5, and more preferably has a molecular weight distribution of 1 to 3. Therefore, when a polymer having a weight average molecular weight and a molecular weight distribution in the ranges described above is used in a photoresist composition, the polymer can exhibit appropriate properties in terms of developability, coatability, and heat resistance.

The polymer according to the present invention having a structure such as described above can be produced by polymerizing an acrylic monomer having the structure of the formula (1) and optionally a (meth)acrylate derivative and a norbornene derivative by a conventional polymerization method, for example, a bulk polymerization method, a solution polymerization method, a suspension polymerization method, a bulk suspension polymerization method, or an emulsion polymerization method.

Preferably, the polymer can be polymerized by radical polymerization, and at this time, any radical polymerization initiator that is usually used as a radical polymerization initiator can be used without any particular limitations. Specifically, any compound selected from the group consisting of azobisisobutyronitrile (AIBN), benzoyl peroxide (BPO), lauryl peroxide, azobisisocapronitrile, azobisisovaleronitrile, t-butyl hydroperoxide, and mixtures thereof can be used. As the polymerization solvent, one or more selected from benzene, toluene, xylene, benzene halide, diethyl ether, tetrahydrofuran, esters, ethers, lactones, ketones, amides, and alcohols can be used.

The polymerization temperature at the time of polymerization reaction is appropriately selected and used in accordance with the type of the catalyst. Furthermore, the molecular weight distribution of the polymer produced can be controlled by appropriately changing the amount of use of the polymerization initiator and the reaction time. After polymerization is completed, it is preferable that unreacted monomers and side products remaining in the reaction mixture be removed by a precipitation method using a solvent.

The polymer according to the present invention obtained by controlling the type and content of the monomer according to the production method described above has excellent film-forming properties, and is effective as a base resin for a resist composition which is excellent in the adhesiveness of the resist to the substrate, non-substrate-dependency, sensitivity, and resolution power. Particularly, the polymer of the present invention is useful as a base resin for a positive chemically amplified resist composition that is exposed by using KrF excimer laser, ArF excimer laser, EUV, X-radiation, an electron beam or the like.

Thus, according to another embodiment of the present invention, a resist composition containing the polymer described above is provided.

More particularly, the resist composition contains the polymer described above, an acid generator, and a solvent.

The polymer is the same as described above, and may be contained in an amount of 3% to 20% by weight relative to the total weight of the resist composition. If the content of the polymer is less than 3% by weight, the viscosity of the composition becomes excessively low so that it is difficult to form a film having a desired thickness, and there is a risk that a severe pattern loss may occur due to the relatively large amount of the acid generator. If the content of the polymer is greater than 20% by weight, the film thickness becomes excessively thick so that radiation transmissivity is decreased, and it may be difficult to obtain a perpendicular pattern.

The acid generator is a photoacid generator (hereinafter, referred to as “PAG”), and an onium salt such as an iodonium salt, a sulfonium salt, a phosphonium salt, a diazonium salt, a pyridinium salt, an imide or the like can be used. Preferably, one or more of sulfonium salts represented by the following formulae (18) and (19) can be used, and more preferably, triphenylsulfonium nonaflate can be used.

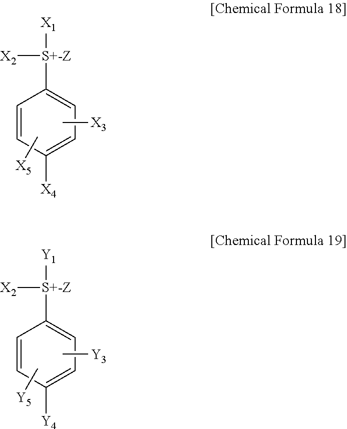

wherein X₁, X₂, Y₁ and Y₂ each independently represent any one selected from the group consisting of a hydrogen atom, an alkyl group having 1 to 10 carbon atoms, an alkenyl group, a haloalkyl group, an aralkyl group having 7 to 30 carbon atoms, an aryl group having 6 to 30 carbon atoms, and combinations thereof, while X₁ and X₂, or Y₁ and Y₂ may be joined together to form a saturated or unsaturated hydrocarbon ring having 3 to 30 carbon atoms. Preferably, X₁, X₂, Y₁ and Y₂ each independently represent any one selected from the group consisting of a hydrogen atom, an alkyl group having 1 to 5 carbon atoms, an allyl group, a perfluoroalkyl group, a benzyl group, an aryl group having 6 to 18 carbon atoms, and combinations thereof, and X₁ and X₂, or Y₁ and Y₂ may be joined together to form a saturated or unsaturated hydrocarbon ring having 3 to 30 carbon atoms.

X₃, X₄, X₅, Y₃, Y₄ and Y₅ each independently represent any one selected from the group consisting of a hydrogen atom, an alkyl group having 1 to 30 carbon atoms, a halogen group, an alkoxy group having 1 to 30 carbon atoms, an aryl group having 6 to 30 carbon atoms, a thiophenoxy group, a thioalkoxy group having 1 to 30 carbon atoms, an alkoxycarbonylalkoxy group having 1 to 20 carbon atoms, and combinations thereof. Preferably, X₃, X₄, X₅, Y₃, Y₄ and Y₅ each independently represent any one selected from the group consisting of a hydrogen atom, an alkyl group having 1 to 20 carbon atoms, a halogen group, an alkoxy group having 1 to 20 carbon atoms, an aryl group having 6 to 18 carbon atoms, a thiophenoxy group, a thioalkoxy group having 1 to 20 carbon atoms, an alkoxycarbonylmethoxy having 1 to 10 carbon atoms, and combinations thereof.

Z of the anion moiety represents OSO₂CF₃, OSO₂C₄F₉, OSO₂C₈F₁₇, N(CF₃)₂, N(C₂F₅)₂, N(C₄F₉)₂, C(CF₃)₃, C(C₂F₅)₃, C(C₄F₉)₃, or a functional group represented by the following formula (20):

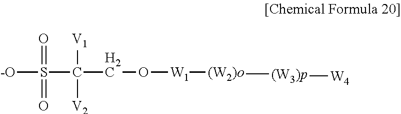

wherein V₁ and V₂ each independently represent a halogen atom;

W₁ represents —C═O— or —(SO₂)—;

W₂ represents an alkanediyl group having 1 to 10 carbon atoms;

W₃ represents any one selected from the group consisting of a cycloalkyl group having 3 to 30 carbon atoms, an aryl group having 6 to 30 carbon atoms, an aralkyl group having 7 to 30 carbon atoms, an aryloxy group having 6 to 30 carbon atoms, an arylthio group having 6 to 30 carbon atoms, and a heterocyclic group having 5 to 30 carbon atoms;

W₄ represents any one selected from the group consisting of a hydrogen atom, a halogen group, an alkyl group having 1 to 10 carbon atoms, an alkoxy group having 1 to 10 carbon atoms, a haloalkyl group having 1 to 10 carbon atoms, an alkylthio group having 1 to 10 carbon atoms, an aryl group having 6 to 30 carbon atoms, and combinations thereof;

o represents an integer of 0 or 1; and

p represents an integer from 0 to 2.

When the acid generator described above is produced by linking a cyclic alkyl group to an anion, the diffusion distance of the acid in the resist film can be appropriately maintained short, high permeability can be exhibited, and as a result, a high resolution resist can be obtained.

Preferably, in the formula (20), A of the anion moiety can be selected from the group consisting of functional groups represented by the following formulae (20-1) to (20-36):

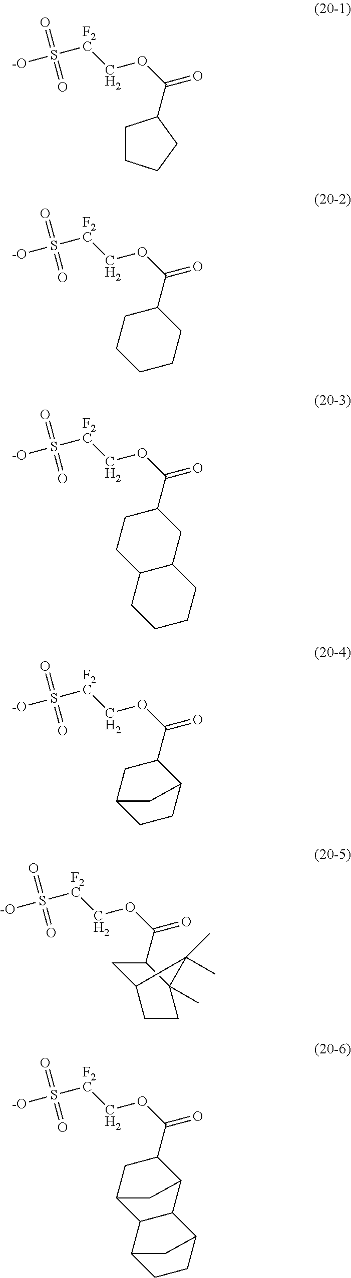

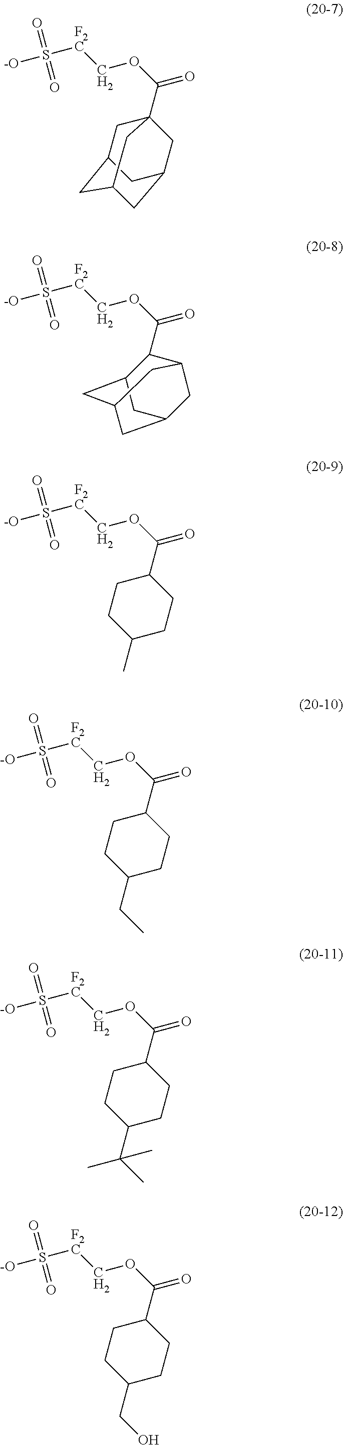

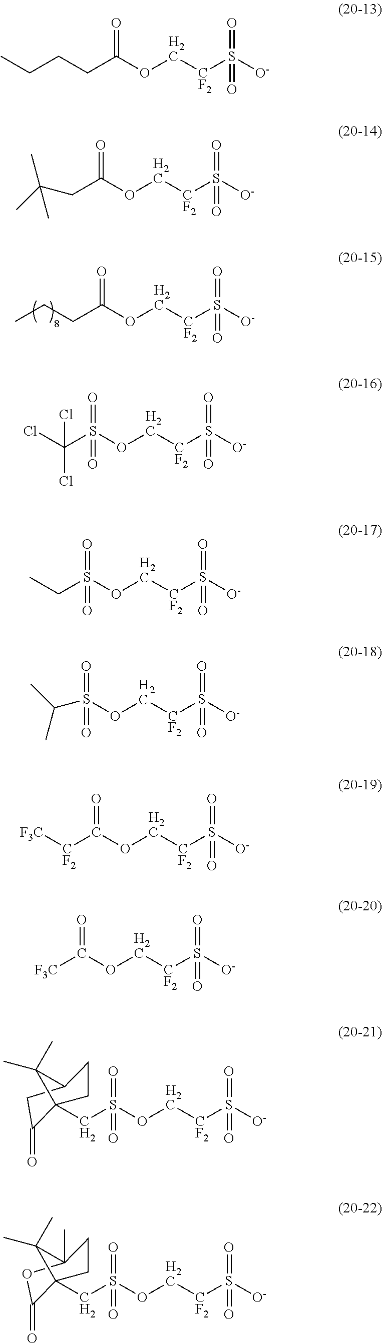

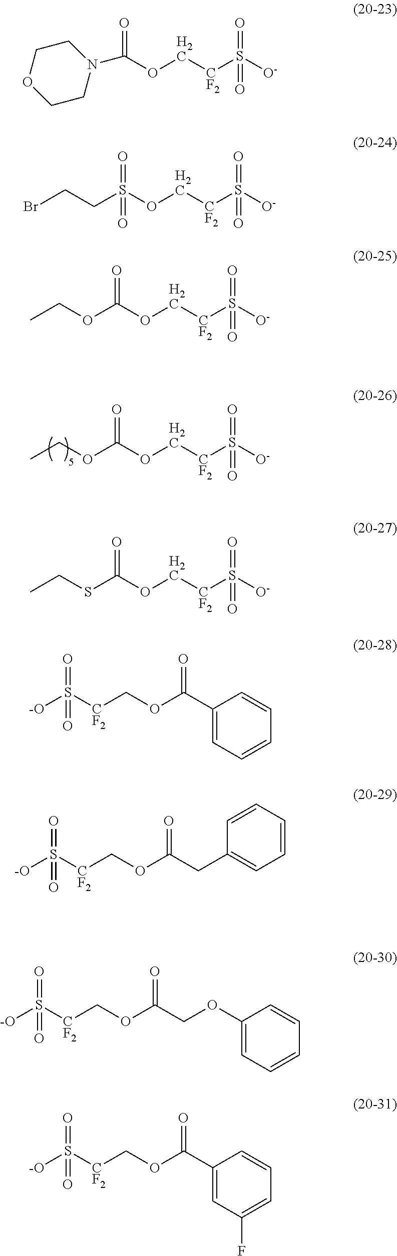

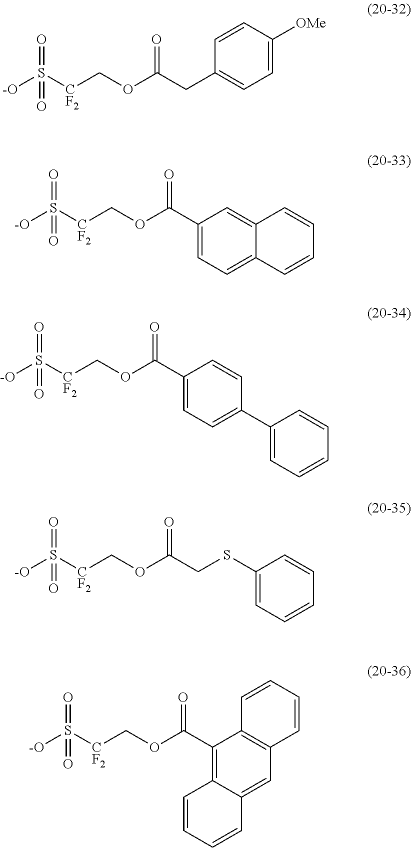

Furthermore, in the formulae (18) and (19), preferred examples of the cation moiety include structures represented by the following formulae (21-1) to (21-16):

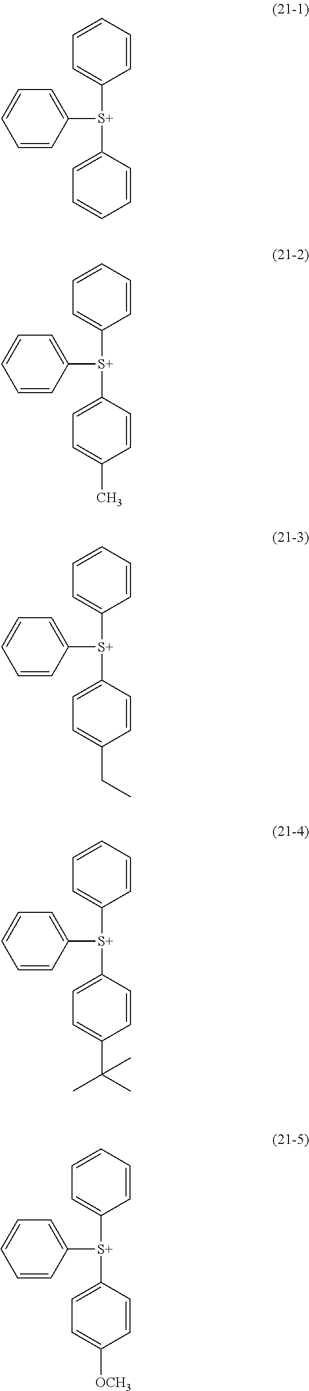 [SMILES: c1ccc([S+](c2ccccc2)c2ccccc2)cc1 ; Cc1ccc([S+](c2ccccc2)c2ccccc2)cc1 ; CCc1ccc([S+](c2ccccc2)c2ccccc2)cc1 ; CC(C)(C)c1ccc([S+](c2ccccc2)c2ccccc2)cc1 ; COc1ccc([S+](c2ccccc2)c2ccccc2)cc1]

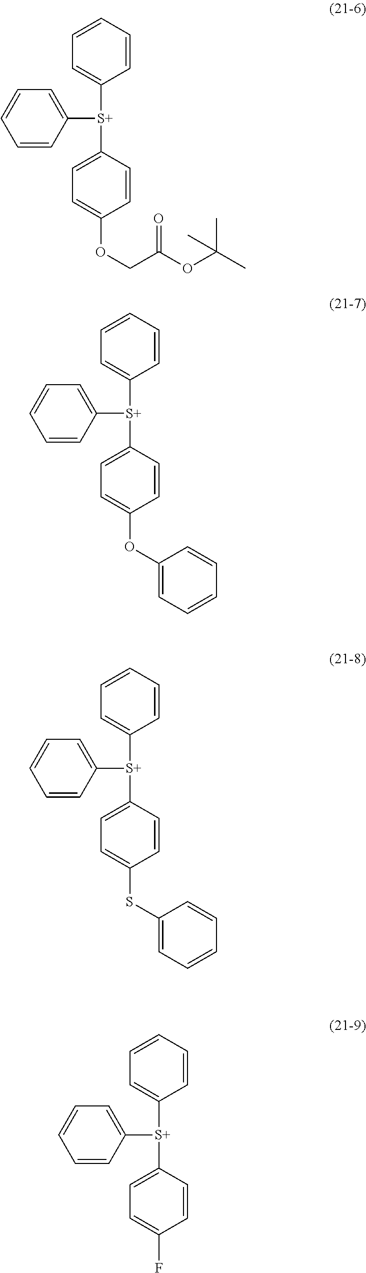 [SMILES: CC(C)(C)OC(=O)COc1ccc([S+](c2ccccc2)c2ccccc2)cc1 ; c1ccc(Oc2ccc([S+](c3ccccc3)c3ccccc3)cc2)cc1 ; c1ccc(Sc2ccc([S+](c3ccccc3)c3ccccc3)cc2)cc1 ; Fc1ccc([S+](c2ccccc2)c2ccccc2)cc1]

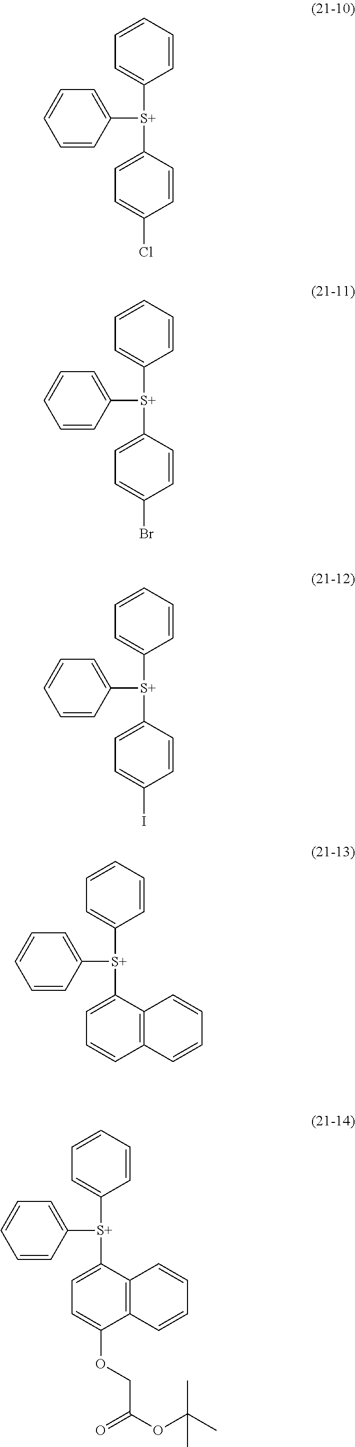 [SMILES: Clc1ccc([S+](c2ccccc2)c2ccccc2)cc1 ; Brc1ccc([S+](c2ccccc2)c2ccccc2)cc1 ; Ic1ccc([S+](c2ccccc2)c2ccccc2)cc1 ; c1ccc([S+](c2ccccc2)c2cccc3ccccc23)cc1 ; CC(C)(C)OC(=O)COc1ccc([S+](c2ccccc2)c2ccccc2)c2ccccc12]

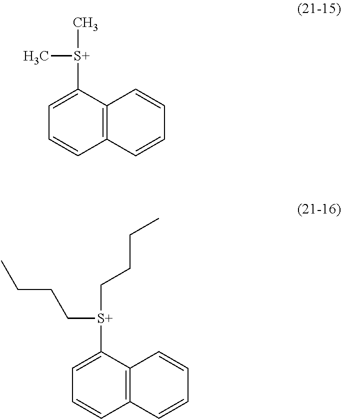 [SMILES: C[S+](C)c1cccc2ccccc12 ; CCCC[S+](CCCC)c1cccc2ccccc12]

The acid generators described above can be used singly, or two or more kinds may be used as a mixture. Furthermore, the acid generator may be incorporated in an amount of 0.3 parts to 15 parts by weight, preferably 0.5 parts to 10 parts by weight, and more preferably 2 parts to 10 parts by weight, relative to 100 parts by weight of the solids content of the polymer. If the content of the acid generator is greater than 15 parts by weight, perpendicularity of the pattern is markedly deteriorated, and if the content is less than 0.3 parts by weight, there is a risk that flexibility of the pattern may be decreased.

In order to obtain a uniform and flat resist coating film, it is preferable to dissolve the polymer and the acid generator in a solvent having an appropriate evaporation rate and viscosity. Examples of the solvent that can be used in the present invention include esters such as ethylene glycol monomethyl ether, ethylene glycol monoethyl ether, ethylene glycol monopropyl ether, methylcellosolve acetate, ethylcellosolve acetate, propylene glycol monomethyl ether acetate, propylene glycol monoethyl ether acetate, and propylene glycol monopropyl ether acetate; and ketones such as methyl isopropyl ketone, cyclohexanone, methyl 2-hydroxypropionate, ethyl 2-hydroxypropionate, 2-heptanone, ethyl lactate, and γ-butyrolactone. These solvents may be used singly, or two or more kinds may be used as a mixture.

For the solvent, the amount of use can be appropriately adjusted depending on the properties of the solvent, namely, volatility, viscosity and the like, so as to form a uniform resist film.

Furthermore, the resist composition according to the present invention may further contain additives according to the purpose of enhancing coatability.

Any additive that is usually applied to resist compositions can be used without any particular limitations, and specific examples include an alkali dissolution inhibitor, an acid diffusion inhibitor, and a surfactant. These additives may be used singly, or as mixtures of two or more kinds.

Any alkali dissolution inhibitor can be applied without any particular limitations as long as it is an alkali dissolution inhibitor conventionally applicable to resist compositions, and specific examples thereof include phenol, and carboxylic acid derivatives.

The acid diffusion inhibitor functions to control the diffusion phenomenon when the acid generated from the acid generator under light irradiation diffuses into the resist film, and to suppress chemical reactions at unexposed areas. When such an acid diffusion inhibitor is used, the storage stability of the radiation-sensitive resin composition can be enhanced, and resolution of the resist can be further enhanced. Furthermore, the change in the line width of the resist pattern due to a fluctuation of the time from exposure to the development treatment (PED) can be suppressed.

As such an acid diffusion inhibitor, a basic compound may be used, and specific examples thereof include amines such as ammonia, methylamine, isopropylamine, n-hexylamine, cyclopentylamine, methylenediamine, ethylenediamine, dimethylamine, diisopropylamine, diethylenediamine, N,N-dimethylmethylenediamine, N,N-dimethylethylenediamine, trimethylamine, triethylamine, N,N,N′,N′-tetramethylmethylenediamine, N,N,N′,N′-tetramethylethylenediamine, N,N,N′,N′-tetramethyltetraethylenepentamine, dimethylethylamine, methylethylpropylamine, benzylamine, phenethylamine, benzyldimethylamine, tetramethylammonium hydroxide, aniline, N,N-dimethyltoluidinetriphenylamine, phenylenediamine, pyrrole, oxazole, isoxazole, thiazole, isothiazole, imidazole, pyrazole, pyrroline, pyrrolidine, imidazoline derivatives, imidazolidine derivatives, pyridine derivatives, pyridazine derivatives, pyrimidine derivatives, pyrazine derivatives, pyrazoline derivatives, pyrazolidine derivatives, piperidine derivatives, piperazine derivatives, and morpholine; nitrogen-containing compounds such as aminobenzoic acid, indolecarboxylic acid, amino acid derivatives (for example, nicotinic acid, alanine, arginine, and aspartic acid), 3-pyridinesulfonic acid, pyridinium p-toluenesulfonate, 2-hydroxypyridine, aminocresol, 2,4-quinolinediol, 2-(2-hydroxyethyl)pyridine, and 1-(2-hydroxyethyl)piperazine; amide derivatives such as formamide, N-methylformamide, N,N-dimethylformamide, acetamide, N-methylacetamide, N,N-dimethylacetamide, propionamide, and benzamide; and imide derivatives such as phthalimide, succinimide, and maleimide.

The acid diffusion inhibitor may be incorporated in an amount of 0.01 parts to 5 parts by weight, and preferably 0.1 parts to 1 part by weight, relative to 100 parts by weight of the polymer solids content. If the content of the acid diffusion inhibitor is less than 0.01 parts by weight, the influence increases with the retention time after exposure, and there is a risk that the pattern shape may be affected. If the content is greater than 5 parts by weight, there is a risk that resolution and sensitivity may decrease.

The surfactant is intended to improve coatability, developability and the like, and specific examples include, but are not limited to, polyoxyethylene lauryl ether, polyoxyethylene stearyl ether, polyoxyethylene, and polyethylene glycol dilaurate.

The resist composition according to the present invention having a composition such as described above exhibits excellent adhesiveness, storage stability, and enhanced line width roughness, and exhibits excellent resolution in both C/H patterns and L/S patterns. Furthermore, the resist composition has an excellent process window, an excellent pattern profile can be obtained regardless of the type of the substrate, and improved contrast is exhibited. Accordingly, the resist composition described above is useful as a positive chemically amplified photoresist composition which responds to radiation such as far-ultraviolet radiation such as KrF excimer laser light, ArF excimer laser, or F2 excimer laser; X-radiation such as synchrotron radiation; or a charged particle beam such as EUV.

According to still another embodiment of the present invention, a method for forming a pattern using the resist composition is provided.

Specifically, the method for forming a pattern includes a step of applying the resist composition described above on a substrate to form a resist film; a step of heat treating the resist film and then exposing the resist film into a predetermined pattern; and then a step of developing the exposed resist pattern.

A wafer substrate may be used as the substrate, and as a method for applying a composition on a substrate, a spin coating method, a flow coating method, or a roll coating method can be used.

Specifically, the resist composition is applied on a substrate such as a silicon wafer to obtain a film thickness of 0.7 μm to 0.1 μm, and this is preliminarily baked for 1 to 10 minutes at 60° C. to 150° C., and preferably for 1 to 5 minutes at 80° C. to 130° C.

Subsequently, the resist film is partially irradiated with a radiation so as to form a fine pattern. The radiation that can be used to this end is not particularly limited, but I-line which is an ultraviolet radiation; KrF excimer laser light, ArF excimer laser light, F₂ excimer laser light, or X-radiation, which are all far-ultraviolet radiations; or an electron beam which is a charged particle radiation, can be used. The radiation can be appropriately selected and used depending on the type of the acid generator.

Specifically, a radiation is irradiated at a dose of about 1 to 200 mJ/cm², and preferably about 10 to 100 mJ/cm², and then the resist film is subjected to post-exposure baking (PEB) for 1 to 5 minutes at 60° C. to 150° C., and preferably for 1 to 3 minutes at 80° C. to 130° C.

After the exposure step, the exposed resist pattern is developed by a conventional method such as an immersion method, a paddle method, or a spray method, using a developer solution for 0.1 to 3 minutes, and thereby, a desired pattern is formed on the substrate. As the developer solution, an aqueous solution containing sodium hydroxide, potassium hydroxide, sodium carbonate, sodium silicate, sodium metasilicate, ammonia, ethylamine, n-propylamine, triethylamine, tetramethylammonium hydroxide, or tetraethylammonium hydroxide can be used, and preferably, it is desirable to use tetramethylammonium hydroxide.

Furthermore, the developer solution may optionally contain additives such as a surfactant and a water-soluble alcohol.

When the method for forming a pattern using the resist composition according to the present invention is used as described above, a fine resist pattern having excellent sensitivity and resolution can be formed.

### EXAMPLES

Hereinafter, the present invention will be described in detail by way of Examples so that a person having ordinary skill in the art to which the present invention is pertained, can easily carry out the invention. However, the present invention can be embodied in various different forms, and is not intended to be limited to the Examples described herein.

### Synthesis Example 1 for Acrylic Monomer

### Preparation of adamantane-1-carboxylic acid 3-methyl-3-(2-methylacryloyloxy)butyl Ester Monomer

### Step 1) Preparation of Intermediate Compound (iii)

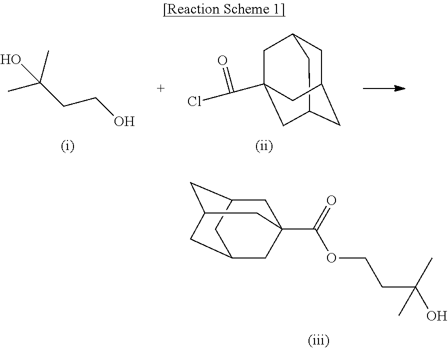

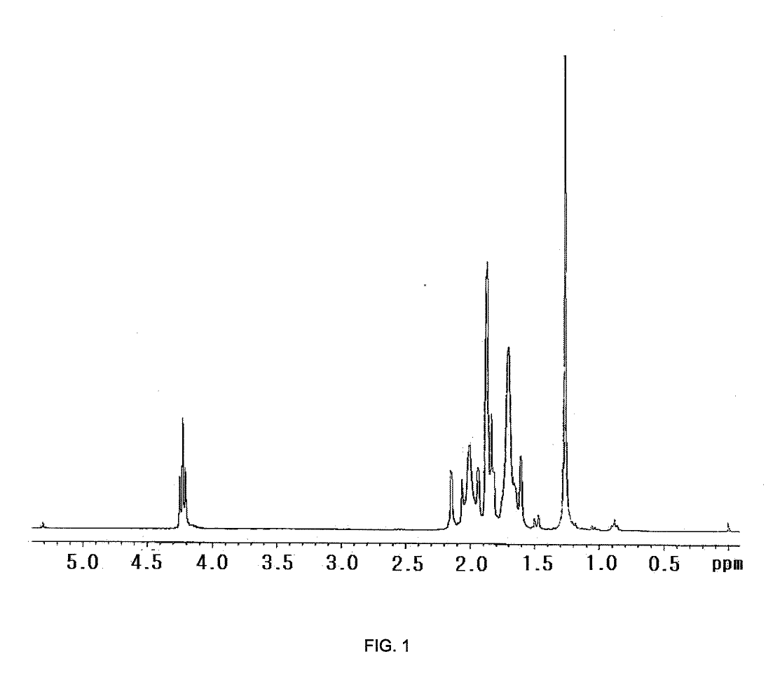

35 mL of triethylamine was added at 0° C. to a solution prepared by dissolving 21.84 g of 3-methyl-1,3-butanediol in 500 mL of methylene chloride, and then 50 g of adamantanecarboyl chloride was slowly added dropwise to the solution. The mixture was stirred for 6 hours at normal temperature. The reaction mixture obtained therefrom was mixed with methylene chloride, subsequently the mixture was washed with acid and distilled water, and only the organic layer was separated. The solvent was removed from the separated organic layer, and thus 51.94 g of an intermediate product (iii) was obtained. The structure of the intermediate product (iii) was confirmed by ¹H-NMR, and the results are presented in FIG. 1.

¹H-NMR: δ(ppm) 1.2(s, 6H), 1.6˜1.8(m, 10), 1.85(t, 3H), 2.0(m, 5H), 4.22(t, 2H)

### Step 2) Preparation of adamantane-1-carboxylic acid 3-methyl-3-(2-methylacryloyloxy)butyl Ester Monomer (1a)

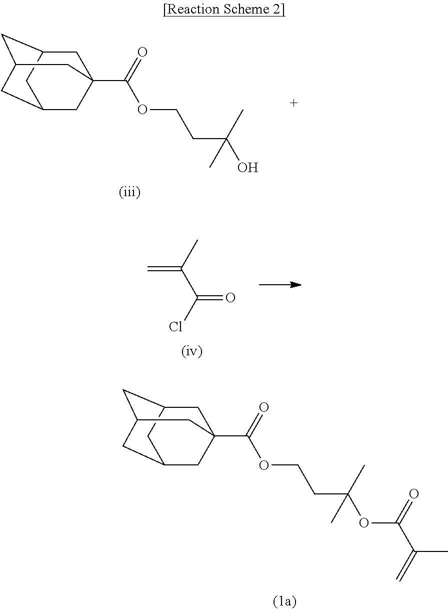

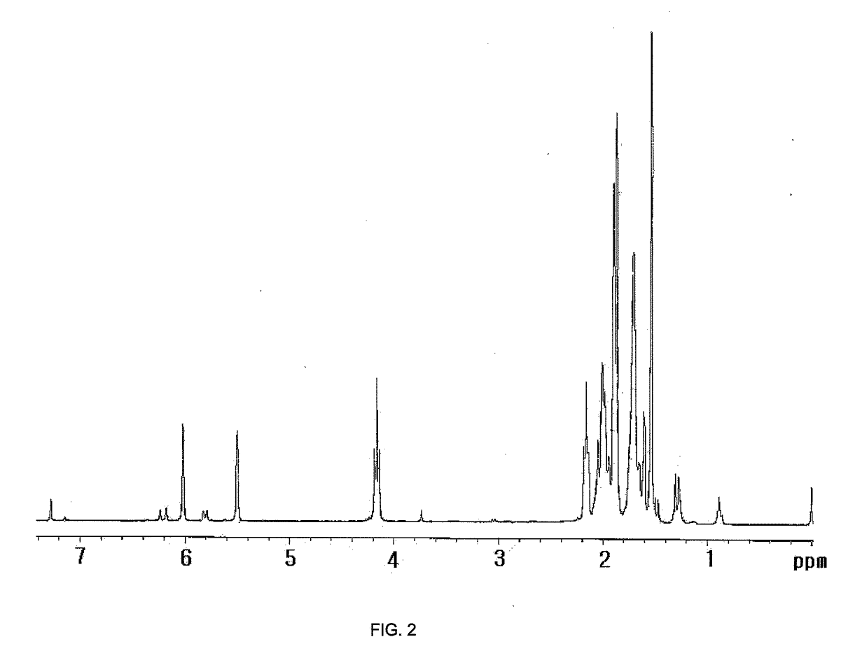

In a double-necked round bottom flask equipped with a stirrer, 48 mL of triethylamine was slowly added dropwise at 0° C. to a solution prepared by dissolving 76 g of the intermediate product (iii) produced in the Step 1, 35.8 g of methacrylic chloride (iv), 6.98 g of N,N-dimethylaminopyridine, and 0.05 g of Irganox™ 1010 (manufactured by BASF GmbH) as an oxidation inhibitor in 500 mL of 1,2-dichloroethane, and then the mixture was stirred for about 8 hours at normal temperature. The reaction mixture thus obtained was acid-treated with 300 mL of a 1% aqueous hydrochloric acid solution, subsequently the reaction mixture was washed with distilled water, and only the organic layer was separated. The solvent was completely removed from the separated organic layer, and thus 69 g of an adamantane-1-carboxylic acid 3-methyl-3-(2-methylacroyloxy)butyl ester monomer of the formula (1a) was obtained. The structure of the compound of the formula (1a) produced as described above was confirmed by ¹H-NMR, and the results are presented in FIG. 2.

¹H-NMR: δ(ppm) 1.57(s, 6H), 1.6˜1.8(m, 10H), 1.85(s, 3H), 1.87(s, 2H), 1.99(m, 3H), 2.15(t, 2H), 4.19(t, 2H), 5.5(s, 1H), 6.0(s, 1H)

### Synthesis Example 2 for Acrylic Monomer

### Preparation of adamantane-1-carboxylic acid 3-methyl-3-acryloyloxybutyl Ester Monomer

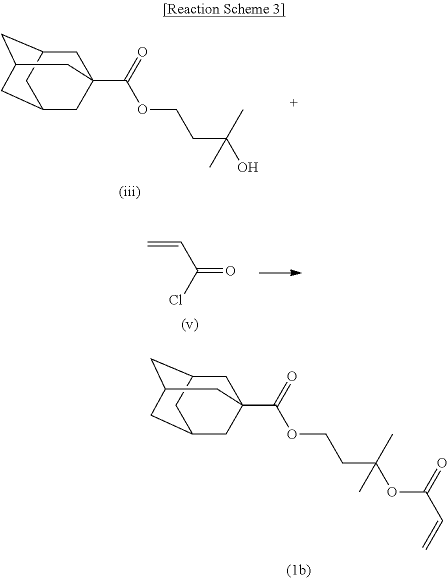

In a double-necked round bottom flask equipped with a stirrer, 48 mL of triethylamine was slowly added dropwise at 0° C. to a solution prepared by dissolving 76 g of the intermediate product (iii) obtained in Step 1 of Synthesis Example 1, 33.8 g of acrylic chloride (V), 6.98 g of N,N-dimethylaminopyridine, and 0.05 g of Irganox™ 1010 (manufactured by BASF GmbH) in 500 mL of 1,2-dichloroethane, and then the mixture was stirred for 8 hours at normal temperature. The reaction mixture thus obtained was washed with distilled water, and then only the organic layer was separated. The solvent was completely removed from the separated organic layer, and thus 66 g of adamantane-l-carboxylic acid 3-methyl-3-acryloyloxybutyl ester monomer of formula (1b) was obtained.

¹H-NMR: δ(ppm) 1.57(s, 6H), 1.6˜1.8(m, 6H), 1.85(s, 3H), 1.87(s, 5H), 2.03(s, 3H), 4.19(t, 2H), 5.95(d, 1H), 6.16(dd, 1H), 6.5(d, 1H)

### Synthesis Example 1 for Polymer

### Synthesis of Polymer (16)

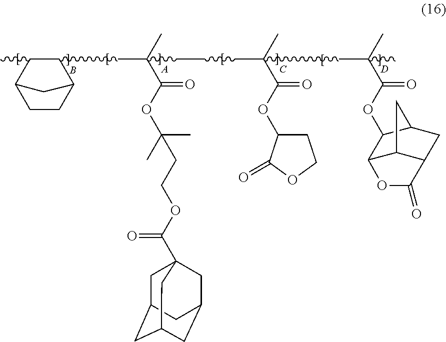

9.04 g of norbornene as a monomer for polymerization, and 3.28 g of dimethyl azobisisobutyrate as a polymerization initiator were placed in a flask together with 55 g of 1,4-dioxane, and the mixture was thoroughly mixed. Subsequently, the interior of the flask was purged with nitrogen gas, and the temperature inside the reactor flask was raised to 70° C. When the internal temperature of the flask reached 70° C., a solution prepared by dissolving 33.4 g of the adamantane-1-carboxylic acid 3-methyl-3-(2-methylacryloyloxy)butyl ester (1a) produced in Synthesis Example 1, 17.5 g of γ-butyrolactyl methacrylate, and 22.2 g of norbornane carbolactone methacrylate in 150 g of 1,4-dioxane was slowly added dropwise to the flask over 2 hours using a syringe pump, and the mixture was allowed to react for 5 hours at the same temperature. After the polymerization reaction was completed, the reaction solution thus obtained was cooled to room temperature. An excess of n-hexane was added to the cooled reaction solution to precipitate out a precipitate, and then the precipitate was separated by filtration. The separated filter cake was washed with the same solvent, and then was dried under reduced pressure. Thus, 70 g of a polymer (16) was obtained. The weight average molecular weight (Mw) of the polymer thus obtained relative to polystyrene standards was 9481 g/mol, and the ratio of the weight average molecular weight and the number average molecular weight (Mw/Mn) was 2.28.

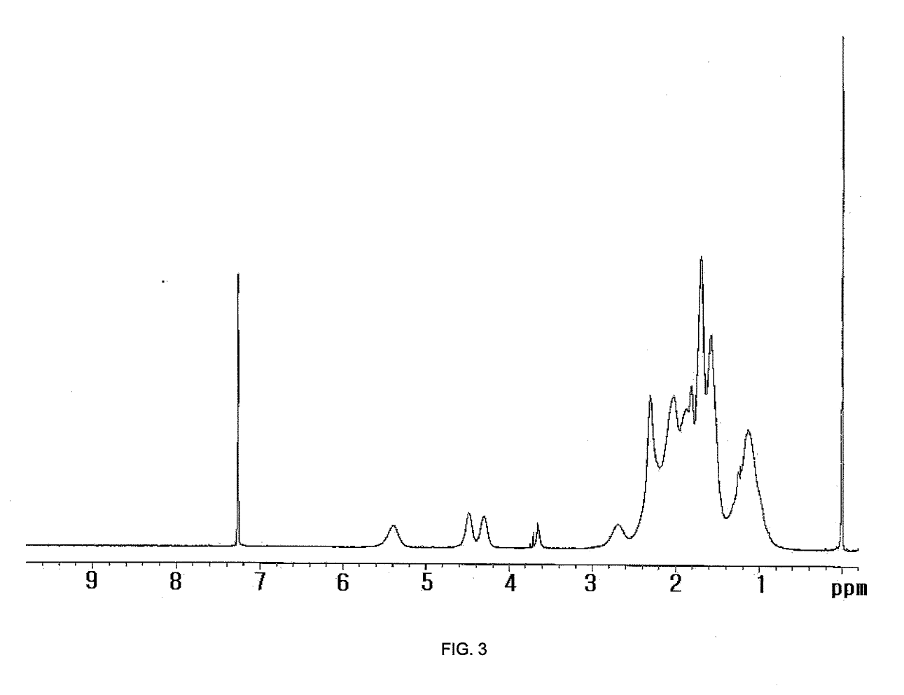

The structure of the polymer (16) produced as described above was confirmed by ¹H-NMR, and the results are presented in FIG. 3.

### Synthesis Example 2 for Polymer

### Synthesis of Polymer (15)

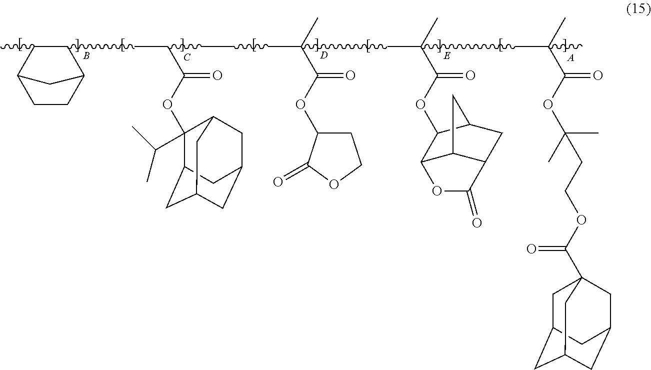

9.04 g of norbornene as a monomer for polymerization, and 5.12 g of dimethyl azobisisobutyrate as a polymerization initiator were placed in a flask together with 60 g of 1,4-dioxane, and the mixture was thoroughly mixed. Subsequently, the interior of the flask was purged with nitrogen gas, and the temperature inside the reactor flask was raised to 70° C. When the internal temperature of the flask reached 70° C., a solution prepared by dissolving 33.4 g of the adamantane-1-carboxylic acid 3-methyl-3-(2-methylacryloyloxy)butyl ester (1a) produced in Synthesis Example 1, 22.2 g of norbornane carbolactone methacrylate, 24.8 g of isopropyladamantane acrylate, and 17.5 g of γ-butyrolactyl methacrylate in 160 g of 1,4-dioxane was slowly added dropwise to the flask over 2 hours using a syringe pump, and the mixture was allowed to react for 5 hours at the same temperature. After the polymerization reaction was completed, the reaction solution thus obtained was cooled to room temperature. An excess of n-hexane was added to the cooled reaction solution to precipitate out a precipitate, and then the precipitate was separated by filtration. The separated filter cake was washed with the same solvent, and then was dried under reduced pressure. Thus, 85 g of a polymer (15) was obtained. The weight average molecular weight (Mw) of the polymer thus obtained relative to polystyrene standards was 6878 g/mol, and the ratio of the weight average molecular weight and the number average molecular weight (Mw/Mn) was 1.70.

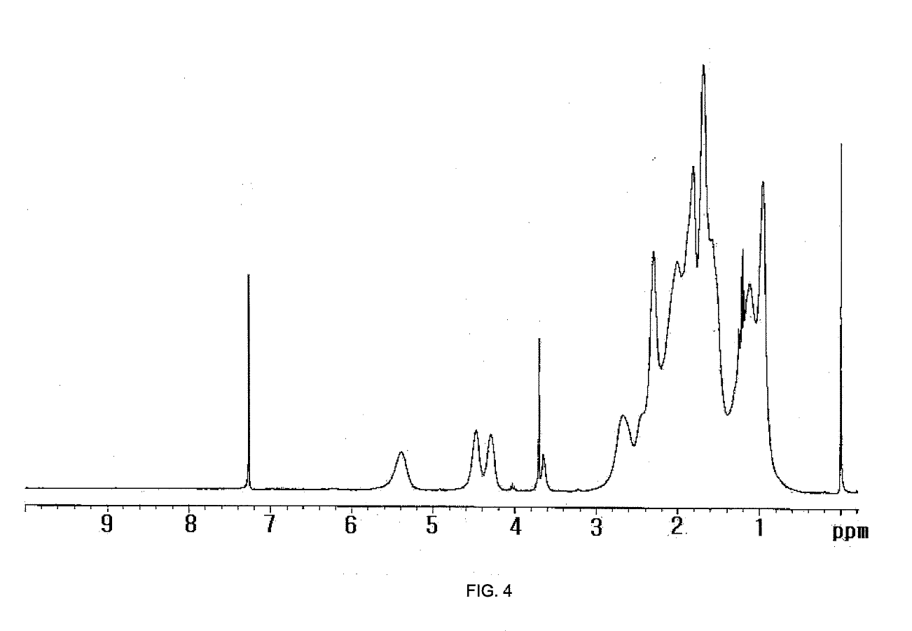

The structure of the polymer (15) produced as described above was confirmed by ¹H-NMR, and the results are presented in FIG. 4.

### Synthesis Example 3 for Polymer

### Synthesis of Polymer (7)

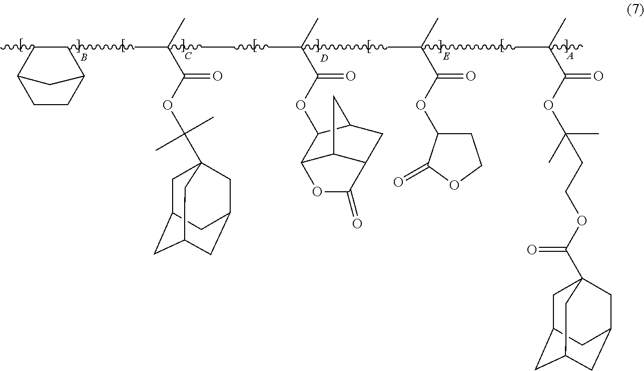

9.04 g of norbornene as a monomer for polymerization, 26.2 g of isopropyladamantane methacrylate, and 5.12 g of dimethyl azobisisobutyrate as a polymerization initiator were placed in a flask together with 72 g of 1,4-dioxane, and the mixture was thoroughly mixed. Subsequently, the interior of the flask was purged with nitrogen gas, and the temperature inside the reactor flask was raised to 70° C. When the internal temperature of the flask reached 70° C., a solution prepared by dissolving 16.7 g of the adamantane-1-carboxylic acid 3-methyl-3-(2-methylacryloyloxy)butyl ester (1a) produced in Synthesis Example 1, 17.5 g of γ-butyrolactyl methacrylate, and 22.2 g of norbornane carbolactone methacrylate in 155 g of 1,4-dioxane was slowly added dropwise to the flask over 2 hours using a syringe pump, and the mixture was allowed to react for 5 hours at the same temperature. After the polymerization reaction was completed, the reaction solution thus obtained was cooled to room temperature. An excess of n-hexane was added to the cooled reaction solution to precipitate out a precipitate, and then the precipitate was separated by filtration. The separated filter cake was washed with the same solvent, and then was dried under reduced pressure. Thus, 35 g of a polymer (7) was obtained. The weight average molecular weight (Mw) of the polymer thus obtained relative to polystyrene standards was 7840 g/mol, and the ratio of the weight average molecular weight and the number average molecular weight (Mw/Mn) was 1.93.

### Synthesis Example 4 for Polymer

### Synthesis of Polymer (13)

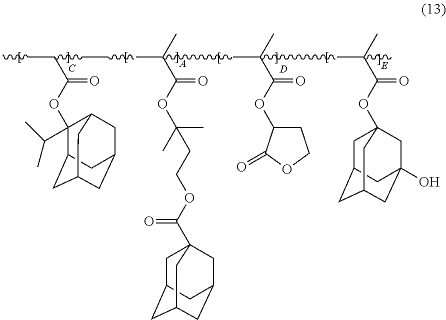

3.63 g of dimethyl azobisisobutyrate as a polymerization initiator was placed in a flask together with 110 g of 1,4-dioxane, and then the mixture was thoroughly mixed. Subsequently, the interior of the flask was purged with nitrogen gas, and the temperature inside the reactor flask was raised to 70° C. When the internal temperature of the flask reached 70° C., a solution prepared by dissolving 16.7 g of the adamantane-1-carboxylic acid 3-methyl-3-(2-methylacryloyloxy)butyl ester produced in Synthesis Example 1, 26.2 g of isopropyladamantane methacrylate, 17.5 g of γ-butyrolactyl methacrylate, and 23.6 g of hydroxyadamantane methacrylate in 200 g of 1,4-dioxane was slowly added dropwise to the flask over 2 hours using a syringe pump, and the mixture was allowed to react for 5 hours at the same temperature. After the polymerization reaction was completed, the reaction solution thus obtained was cooled to room temperature. An excess of n-hexane was added to the cooled reaction solution to precipitate out a precipitate, and then the precipitate was separated by filtration. The separated filter cake was washed with the same solvent, and then was dried under reduced pressure. Thus, 65 g of a polymer (13) was obtained. The weight average molecular weight (Mw) of the polymer thus obtained relative to polystyrene standards was 8011 g/mol, and the ratio of the weight average molecular weight and the number average molecular weight (Mw/Mn) was 2.01.

### Synthesis Example 5 for Polymer

### Synthesis of Polymer (10)

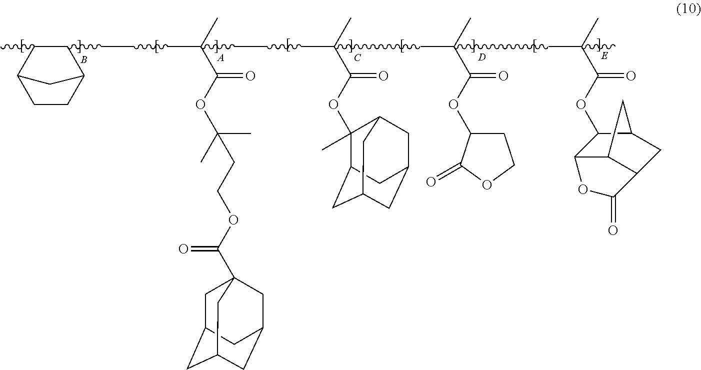

9.04 g of norbornene as a monomer for polymerization, and 3.28 g of dimethyl azobisisobutyrate as a polymerization initiator were placed in a flask together with 55 g of 1,4-dioxane, and the mixture was thoroughly mixed. Subsequently, the interior of the flask was purged with nitrogen gas, and the temperature inside the reactor flask was raised to 70° C. When the internal temperature of the flask reached 70° C., a solution prepared by dissolving 16.7 g of the adamantane-1-carboxylic acid 3-methyl-3-(2-methylacryloyloxy)butyl ester (1a) produced in Synthesis Example 1, 13.2 g of methyladamantane methacrylate, 17.5 g of γ-butyrolactyl methacrylate, and 22.2 g of norbornane carbolactone methacrylate in 155 g of 1,4-dioxane was slowly added dropwise to the flask over 2 hours using a syringe pump, and the mixture was allowed to react for 5 hours at the same temperature. After the polymerization reaction was completed, the reaction solution thus obtained was cooled to room temperature. An excess of n-hexane was added to the cooled reaction solution to precipitate out a precipitate, and then the precipitate was separated by filtration. The separated filter cake was washed with the same solvent, and then was dried under reduced pressure. Thus, 55 g of a polymer (10) was obtained. The weight average molecular weight (Mw) of the polymer thus obtained relative to polystyrene standards was 11754 g/mol, and the ratio of the weight average molecular weight and the number average molecular weight (Mw/Mn) was 2.28.

### Example 1

### Preparation of Resist Composition

A mixture prepared by mixing 100 parts by weight of the polymer (16) produced in Synthesis Example 1 for polymer, with 4 parts by weight of triphenylsulfonium nonaflate as an acid generator, 0.2 parts by weight of tetramethylammonium hydroxide as an acid diffusion inhibitor, and 1,000 parts by weight of propylene glycol methyl ether acetate, was filtered through a 0.2-μm membrane filter, and thus a resist composition was prepared.

### Examples 2 to 8

### Preparation of Resist Composition

Resist compositions were prepared in the same manner as in Example 1, except that the compositions indicated in the following Table 1 were used.

TABLE 1

| | | |
|---|---|---|
| Polymer | Acid | Basic |

| Example No. | Type | Content | generator⁽¹⁾ | additive⁽²⁾ |
|---|---|---|---|---|
| Example 1 | Formula 16 | 100 | 4 | 0.2 |
| Example 2 | Formula 7 | 100 | 5 | 0.3 |
| Example 3 | Formula 13 | 100 | 5 | 0.3 |
| Example 4 | Formula 10 | 100 | 5 | 0.3 |
| Example 5 | Formula 7 | 100 | 6 | 0.3 |
| Example 6 | Formula 13 | 100 | 6 | 0.3 |
| Example 7 | Formula 10 | 100 | 6 | 0.3 |
| Example 8 | Formula 7 | 100 | 6 | 0.4 |
| Example 9 | Formula 13 | 100 | 6 | 0.4 |
| Example 10 | Formula 10 | 100 | 6 | 0.4 |

(unit: parts by weight)
⁽¹⁾Triphenylsulfonium nonaflate
⁽²⁾Tetramethylammonium hydroxide

### Comparative Examples 1 to 3

### Preparation of Resist Composition

Resist compositions were prepared in the same manner as in Example 1, except that the compositions indicated in the following Table 2 were used.

TABLE 2

| Comparative | Polymer | | Acid | Basic |
|---|---|---|---|---|
| Example No. | Type | Content | generator⁽²⁾ | additive⁽³⁾ |
| Comparative | COMA resin⁽¹⁾ | 100 | 2.5 | 0.02 |
| Example 1 | | | | |
| Comparative | COMA resin⁽¹⁾ | 100 | 3.0 | 0.02 |
| Example 2 | | | | |
| Comparative | COMA resin⁽¹⁾ | 100 | 3.0 | 0.03 |
| Example 3 | | | | |

(unit: parts by weight)
⁽¹⁾COMA resin (manufactured by Kumho Petrochemical Co., Ltd.) 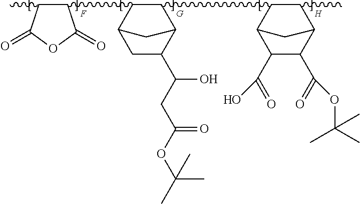
⁽²⁾Triphenylsulfonium nonaflate
⁽³⁾Tetramethylammonium hydroxide

### Test Example

### Performance Evaluation on Resist Pattern

Each of the resist compositions prepared in Examples 1 to 10 and Comparative Examples 1 to 3 as described above was applied on a substrate using a spinner, and was dried at 110° C. for 90 seconds to thereby form a resist film having a thickness of 0.2 μm. The coating film thus formed was exposed using an ArF excimer laser stepper (numerical aperture of the lens: 0.78), and then the exposed film was heat treated for 90 seconds at 110° C. Subsequently, the exposed film was developed with a 2.38 wt % aqueous solution of tetramethylammonium hydroxide for 40 seconds, and was washed and dried. Thus, a positive resist pattern was formed.

For the resist patterns thus formed, the developability with an aqueous solution of tetramethylammonium hydroxide, and the adhesiveness to the substrate, sensitivity and resolution of the resist patterns were measured. The results are presented in Table 3.

In the case of developability, the evaluation was carried out on the basis of the following criteria.

◯: Excellent developability is exhibited without any deformation of the pattern

Δ: Partial deformation of the pattern occurred, but generally satisfactory developability is exhibited.

×: Poor developability is exhibited, with severe deformation of the pattern.

In the case of adhesiveness, the adhesion state of a 0.07-μm line-and-space (L/S) pattern formed after development was observed, and an evaluation was carried out on the basis of the following criteria.

◯: Excellent adhesiveness

Δ: Generally satisfactory adhesiveness

×: Poor adhesiveness

In the case of sensitivity, the amount of exposure that forms a 0.07-μm line-and-space (L/S) pattern formed after development at a line width of 1:1, was defined as the optimum amount of exposure, and this optimum amount of exposure was designated as sensitivity. Furthermore, the minimum pattern dimension that is resolved was designated as resolution.

TABLE 3

| | | | Sensitivity | Resolution |
|---|---|---|---|---|
| | Developability | Adhesiveness | (mJ/cm²) | (nm) |
| Example 1 | ◯ | ◯ | 18 | 70 |
| Example 2 | ◯ | ◯ | 117 | 70 |
| Example 3 | ◯ | ◯ | 18 | 60 |
| Example 4 | ◯ | ◯ | 18 | 90 |
| Example 5 | ◯ | ◯ | 15 | 80 |
| Example 6 | ◯ | ◯ | 16 | 60 |
| Example 7 | ◯ | ◯ | 15 | 80 |
| Example 8 | ◯ | ◯ | 19 | 70 |
| Example 9 | ◯ | ◯ | 18 | 80 |
| Example 10 | ◯ | ◯ | 20 | 90 |
| Comparative | — | — | 16 | 120 |
| Example 1 | | | | |
| Comparative | — | — | 14 | 110 |
| Example 2 | | | | |
| Comparative | — | — | 16.5 | 120 |
| Example 3 | | | | |

* In the Table 3, “—” means that no measurement was made.

The resist compositions of Examples 1 to 10 containing the polymer according to the present invention exhibited excellent developability and adhesiveness, and the resist films produced by using these resist compositions exhibited markedly improved characteristics in terms of sensitivity and resolution, as compared with Comparative Examples 1 to 3.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

1. An acrylic monomer having a structure represented by the following formula (1): 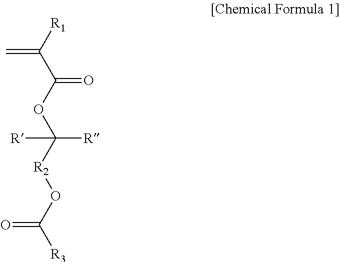
wherein R₁ represents a hydrogen atom or a methyl group;
R₂ represents an alkanediyl group having 1 to 10 carbon atoms;
R₃ represents a cycloalkyl group having 3 to 30 carbon atoms; and
R′ and R″ each independently represent any one selected from the group consisting of a hydrogen atom, an alkyl group having 1 to 10 carbon atoms, a cycloalkyl group having 3 to 30 carbon atoms, and a (C₁-C₁₀ alkyl)cycloalkyl group, provided that R′ and R″ are not hydrogen atoms at the same time.

2. The acrylic monomer according to claim 1, wherein R₂ is selected from the group consisting of methylene, ethylidene, propylidene, trimethylene, tetramethylene, pentamethylene, hexamethylene, and heptamethylene.

3. The acrylic monomer according to claim 1, wherein R₃ is selected from the group consisting of a monocyclic cycloalkyl group having 3 to 14 carbon atoms, a bicyclic cycloalkyl group having 4 to 20 carbon atoms, a tricyclic cycloalkyl group having 10 to 30 carbon atoms, and a tetracyclic cycloalkyl group having 10 to 30 carbon atoms.

4. The acrylic monomer according to claim 1, having a structure represented by the following formula (1a): 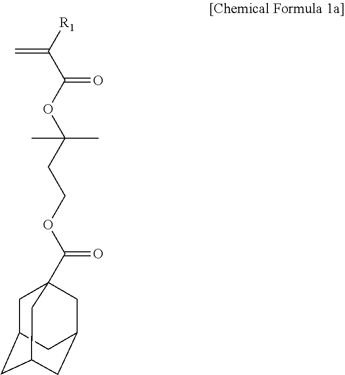
wherein R₁ represents a hydrogen atom or a methyl group.

5. A polymer comprising a repeating unit represented by the following formula (2): 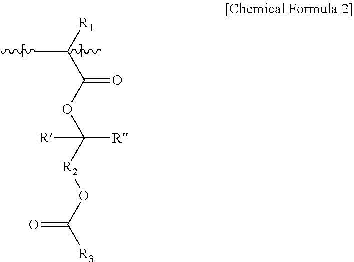
wherein R₁ represents a hydrogen atom or a methyl group;
R₂ represents an alkanediyl group having 1 to 10 carbon atoms;
R₃ represents a cycloalkyl group having 3 to 30 carbon atoms; and
R′ and R″ each independently represent any one selected from the group consisting of a hydrogen atom, an alkyl group having 1 to 10 carbon atoms, a cycloalkyl group having 3 to 30 carbon atoms, and a (C₁-C₁₀ alkyl)cycloalkyl group, provided that R′ and R″ are not hydrogen atoms at the same time.

6. The polymer according to claim 5, wherein R₂ is selected from the group consisting of methylene, ethylidene, propylidene, trimethylene, tetramethylene, pentamethylene, hexamethylene, and heptamethylene.

7. The polymer according to claim 5, wherein R₃ is selected from the group consisting of a monocyclic cycloalkyl group having 3 to 14 carbon atoms, a bicyclic cycloalkyl group having 4 to 20 carbon atoms, a tricyclic cycloalkyl group having 10 to 30 carbon atoms, and a tetracyclic cycloalkyl group having 10 to 30 carbon atoms.

8. The polymer according to claim 5, comprising a repeating unit represented by the following formula (2a): 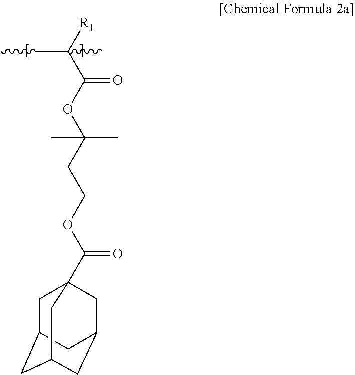
wherein R₁ represents a hydrogen atom or a methyl group.

9. The polymer according to claim 5, further comprising one or more repeating units selected from the group consisting of repeating units represented by the following formulae (3) and (4): 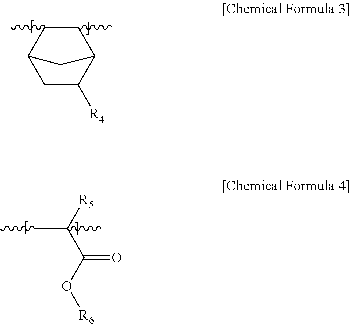
wherein in the above formulae,
R₄ and R₆ each independently represent any one selected from the group consisting of a hydrogen atom, an alkyl group having 1 to 10 carbon atoms, a heteroalkyl group having 1 to 10 carbon atoms, a cycloalkyl group having 3 to 30 carbon atoms, an aryl group having 6 to 30 carbon atoms, a heterocyclic group having 2 to 30 carbon atoms, and combinations thereof; and
R₅ represents any one selected from the group consisting of a hydrogen atom, an alkyl group having 1 to 10 carbon atoms, a perfluoroalkyl group having 1 to 10 carbon atoms, and an alkoxy group having 1 to 10 carbon atoms.

10. The polymer according to claim 5, wherein the polymer is represented by the following formula (5): 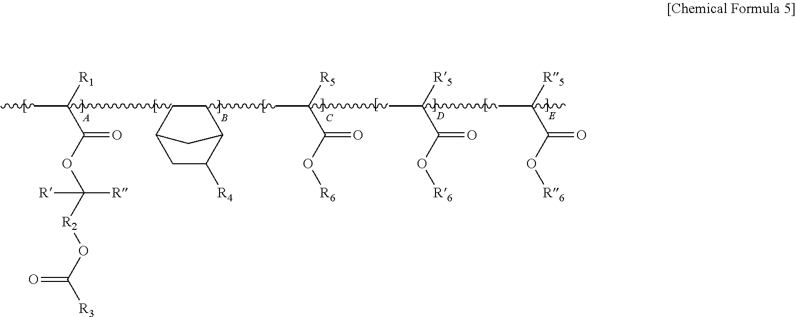
wherein R₁ represents a hydrogen atom or a methyl group;
R₂ represents an alkanediyl group having 1 to 10 carbon atoms;
R₃ represents a cycloalkyl group having 3 to 30 carbon atoms;
R₄ represents any one selected from the group consisting of a hydrogen atom, an alkyl group having 1 to 10 carbon atoms, a heteroalkyl group having 1 to 10 carbon atoms, a cycloalkyl group having 3 to 30 carbon atoms, an aryl group having 6 to 30 carbon atoms, a heterocyclic group having 2 to 30 carbon atoms, and combinations thereof;
R₅, R₅′ and R₅″ each independently represent any one selected from the group consisting of a hydrogen atom, an alkyl group having 1 to 10 carbon atoms, a perfluoroalkyl group having 1 to 10 carbon atoms, and an alkoxy group having 1 to 10 carbon atoms;
R₆, R₆′ and R₆″ each independently represent a hydrogen atom, an alkyl group having 1 to 10 carbon atoms, a heteroalkyl group having 1 to 10 carbon atoms, a cycloalkyl group having 3 to 30 carbon atoms, an aryl group having 6 to 30 carbon atoms, a heterocyclic group having 2 to 30 carbon atoms, and combinations thereof;
R′ and R″ each independently represent any one selected from the group consisting of a hydrogen atom, an alkyl group having 1 to 10 carbon atoms, a cycloalkyl group having 3 to 30 carbon atom, and a (C₁-C₁₀ alkyl)cycloalkyl group, provided that R′ and R″ are not hydrogen atoms at the same time; and
A, B, C, D and E respectively represent the numbers indicating the proportions of the corresponding repeating units in the main chain, and A, B, C, D and E are related such that A+B+C+D+E=1, 0≦A/(A+B+C+D+E)<0.4, 0<B/(A+B+C+D+E)<0.4, 0<C/(A+B+C+D+E)<0.6, 0≦D/(A+B+C+D+E)<0.6, and 0≦E/(A+B+C+D+E)<0.6.

11. The polymer according to claim 5, comprising the repeating unit represented by the formula (2) at a content of 10 mol % to 40 mol %.

12. The polymer according to claim 5, wherein the polymer is selected from the group consisting of polymers represented by the following formulae (6) to (17): 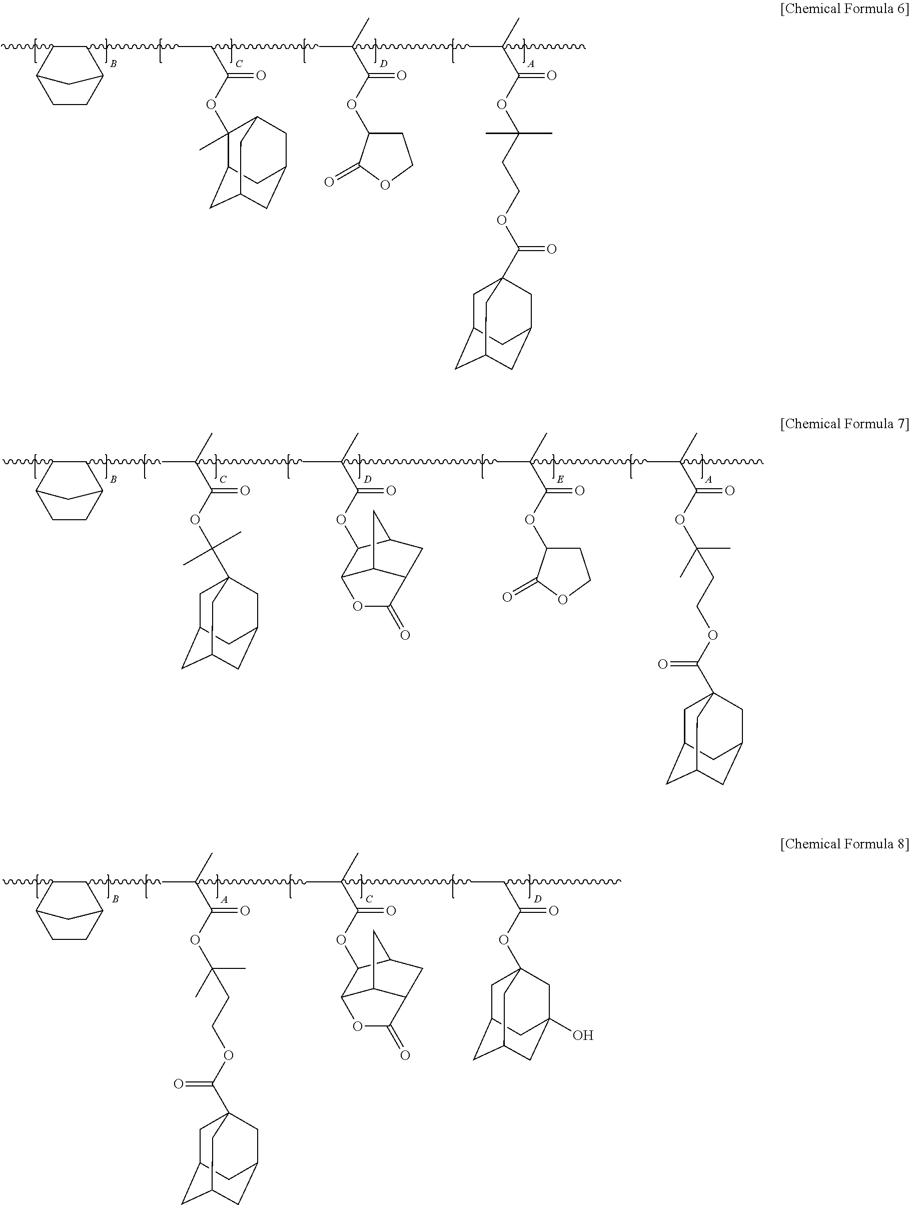 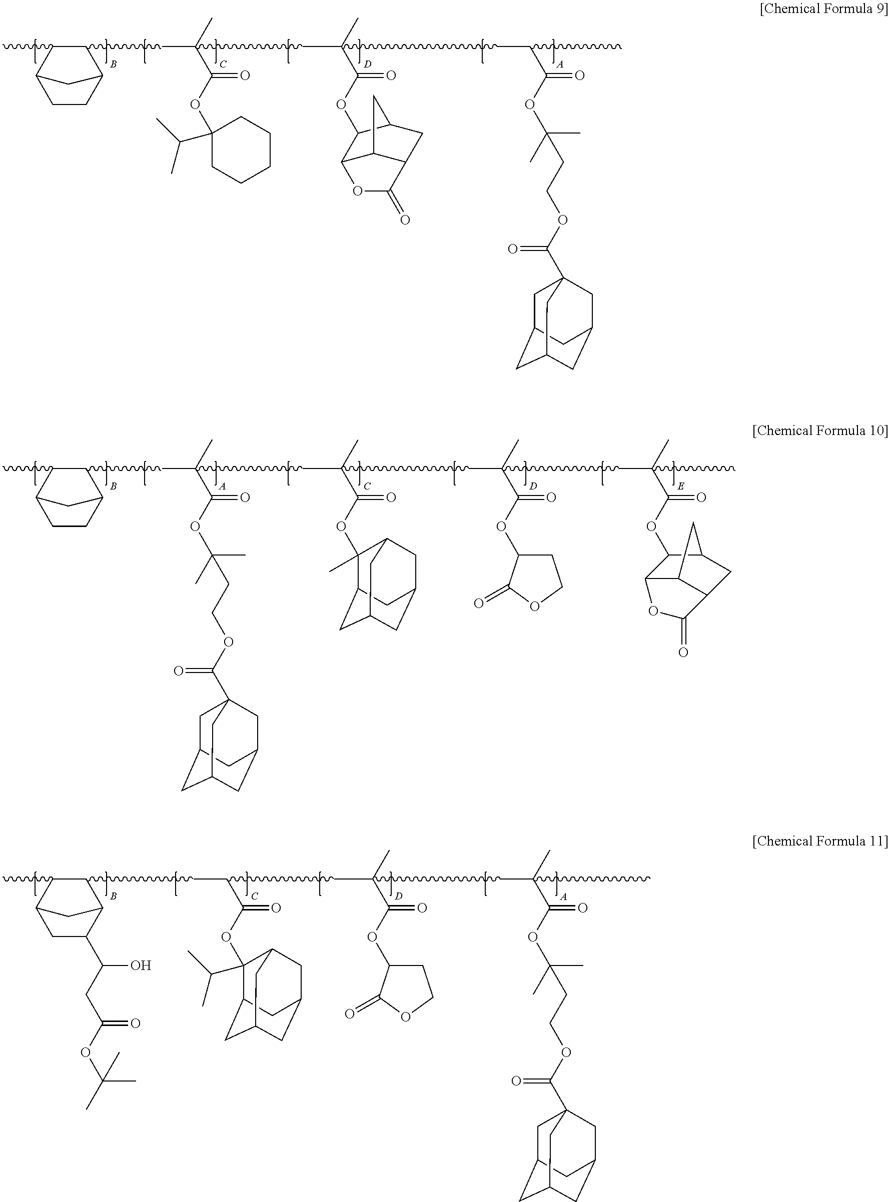 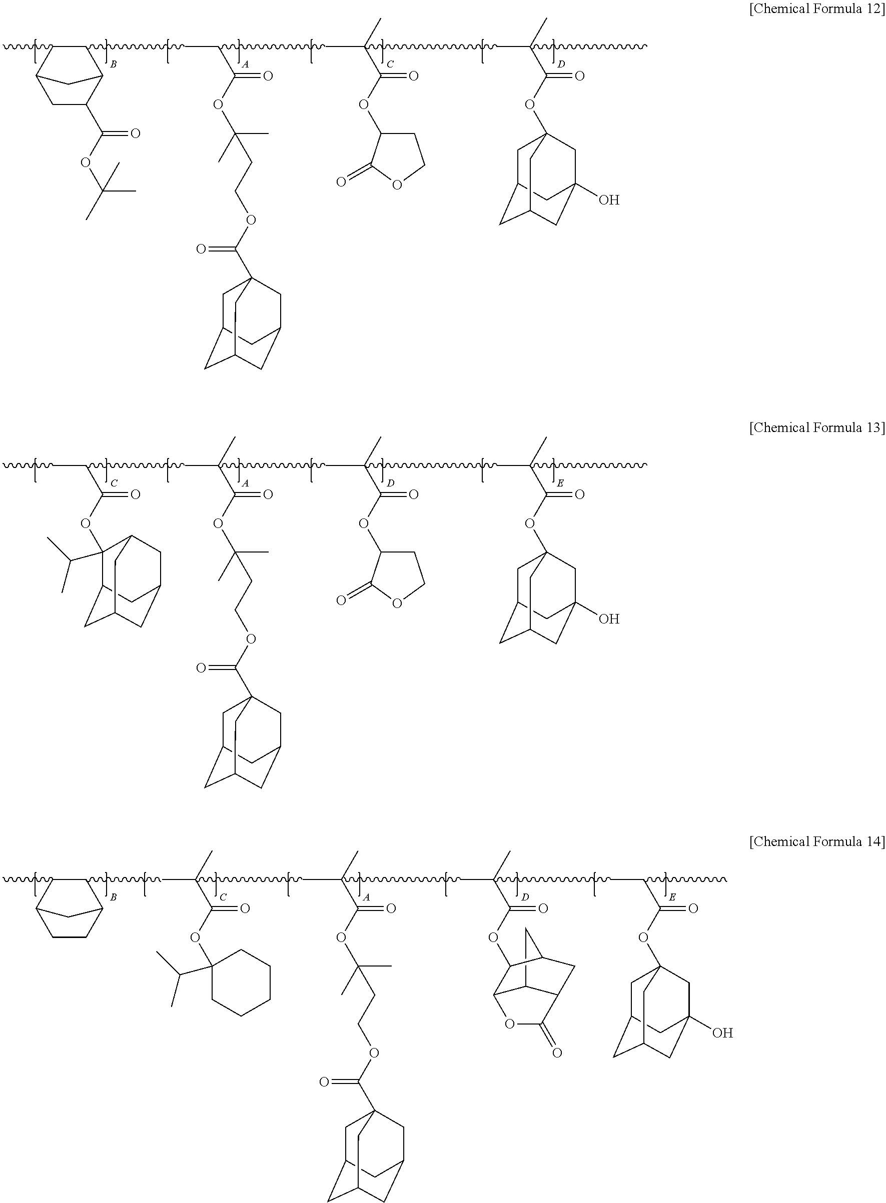 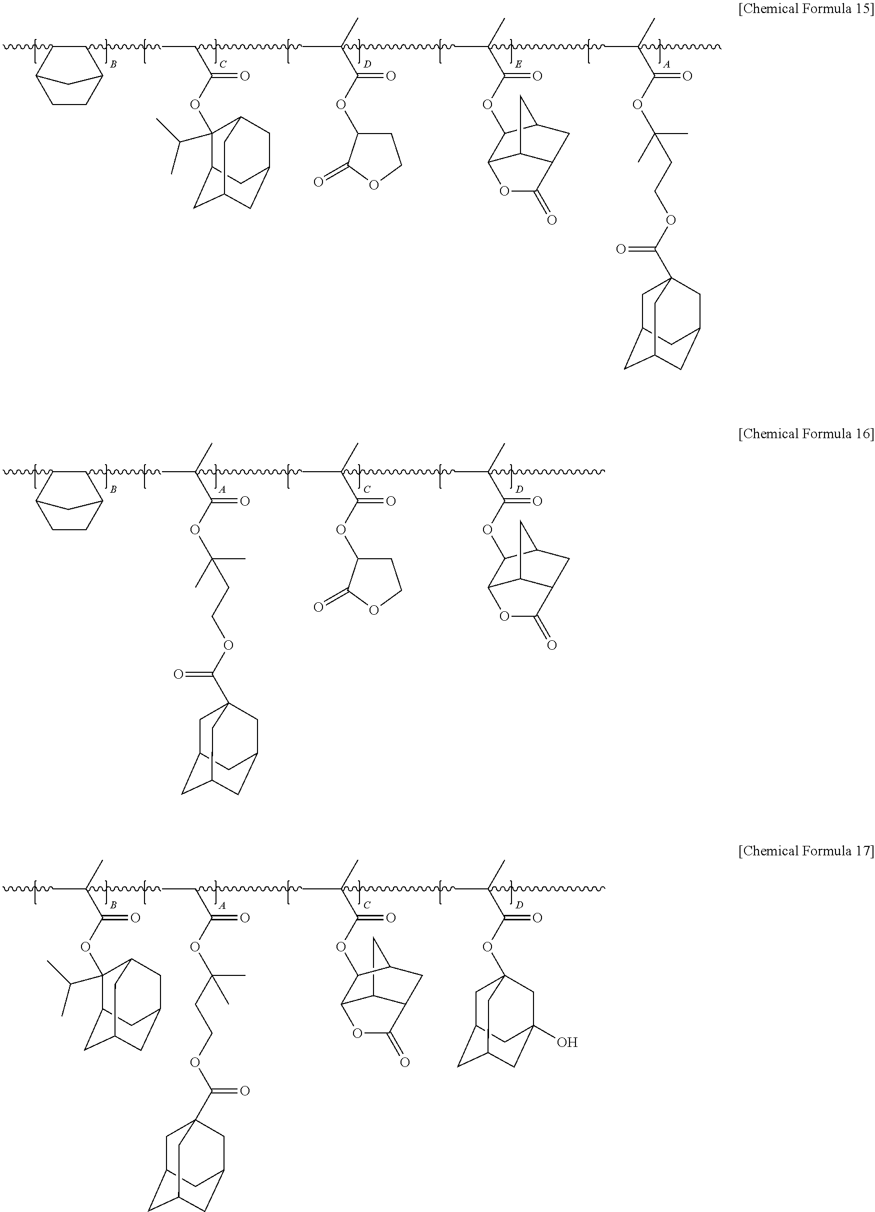
wherein in the above formulae,
A, B, C, D and E respectively represent the numbers indicating the proportions of the corresponding repeating units in the main chain, and A, B, C, D and E are related such that A+B+C+D+E=1, 0≦A/(A+B+C+D+E)<0.4, 0<B/(A+B+C+D+E)<0.4, 0<C/(A+B+C+D+E)<0.6, 0≦D/(A+B+C+D+E)<0.6, and 0≦E/(A+B+C+D+E)<0.6.

13. The polymer according to claim 5, wherein the polymer has a weight average molecular weight of 2,000 g/mol to 1,000,000 g/mol as determined by gel permeation chromatography and calculated relative to polystyrene standards, and may have a molecular weight distribution of 1 to 5.

14. A resist composition comprising the polymer according to claim 5, an acid generator, and a solvent.

15. The resist composition according to claim 14, wherein the resist composition contains the polymer at a content of 3 wt % to 20 wt % relative to the total weight of the resist composition.

16. The resist composition according to claim 14, wherein the acid generator is one or more compounds selected from the group consisting of compounds represented by the following formulae (18) and (19): 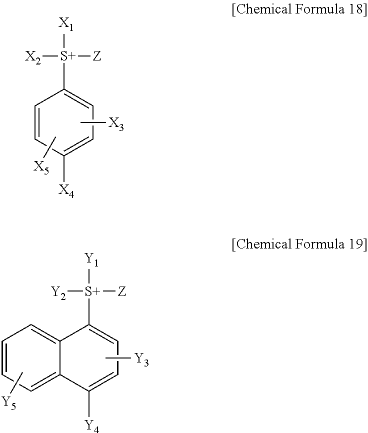
wherein X₁, X₂, Y₁ and Y₂ each independently represent any one selected from the group consisting of a hydrogen atom, an alkyl group having 1 to 10 carbon atoms, an alkenyl group, a haloalkyl group having 1 to 10 carbon atoms, an aralkyl group having 6 to 30 carbon atoms, an aryl group having 6 to 30 carbon atoms, and combinations thereof, while X₁ and X₂, or Y₁ and Y₂ may be joined together to form a saturated or unsaturated hydrocarbon ring having 3 to 30 carbon atoms;
X₃, X₄, X₅, Y₃, Y₄ and Y₅ each independently represent any one selected from the group consisting of a hydrogen atom, an alkyl group having 1 to 30 carbon atoms, a halogen group, an alkoxy group having 1 to 30 carbon atoms, an aryl group having 6 to 30 carbon atoms, a thiophenoxy group, a thioalkoxy group having 1 to 30 carbon atoms, an alkoxycarbonylalkoxy group having 1 to 20 carbon atoms, and combinations thereof;
Z of the anion moiety represents OSO₂CF₃, OSO₂C₄F₉, OSO₂C₈F₁₇, N(CF₃)₂, N(C₂F₅)₂, N(C₄F₉)₂, C(CF₃)₃, C(C₂F₅)₃, C(C₄F₉)₃, or the following formula (20): 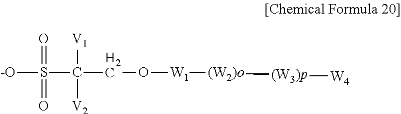
wherein V₁ and V₂ each independently represent a halogen atom;
W₁ represents —(C═O)— or —(SO₂)—;
W₂ represents an alkanediyl group having 1 to 10 carbon atoms;
W₃ represents any one selected from the group consisting of a cycloalkyl group having 3 to 30 carbon atoms, an aryl group having 6 to 30 carbon atoms, an aralkyl group having 7 to 30 carbon atoms, an aryloxy group having 6 to 30 carbon atoms, an arylthio group having 6 to 30 carbon atoms, a heterocyclic group having 5 to 30 carbon atoms, and combinations thereof;
W₄ represents any one selected from the group consisting of a hydrogen atom, a halogen group, an alkyl group having 1 to 10 carbon atoms, an alkoxy group having 1 to 10 carbon atoms, a haloalkyl group having 1 to 10 carbon atoms, an alkylthio group having 1 to 10 carbon atoms, an aryl group having 6 to 30 carbon atoms, and combinations thereof;
o represents an integer of 0 or 1; and
p represents an integer from 0 to 2.

17. The resist composition according to claim 14, wherein the resist composition contains the acid generator at a content of 0.3 to 10 parts by weight relative to 100 parts by weight of the solids content of the resist composition.

18. The resist composition according to claim 14, further comprising an additive selected from the group consisting of an alkali dissolution inhibitor, an acid diffusion inhibitor, a surfactant, a sensitizer, and mixtures thereof.

19. A method for forming a resist pattern, the method comprising:
applying the resist composition according to claim 14 on a substrate to form a resist film;
heat treating the resist film, and then exposing the resist film into a predetermined pattern; and
developing the exposed resist pattern.

20. The method for forming a resist according to claim 19, wherein the exposure process is carried out by using a light source selected from the group consisting of KrF excimer laser light, ArF excimer laser light, extreme ultraviolet laser light, X-radiation, and an electron beam.
